(19) United States
(12) Patent Application Publication
(10) Pub. No.: US 2011/0264034 A1
(43) Pub. Date: Oct. 27, 2011

(54) MEDICAL THERAPY MODIFICATION AUTHORIZATION TECHNIQUES

(75) Inventors: Earle T. Roberts, Maple Grove, MN (US); Irfan Z. Ali, Woodbury, MN (US); Emem D. Akpan, Coon Rapids, MN (US); Scott L. Kalpin, Harris, MN (US); Varaz Shahmirian, Northridge, CA (US); William J. Mitchell, Eden Prairie, MN (US)

(73) Assignee: Medtronic, Inc., Minneapolis, MN (US)

(21) Appl. No.: 12/768,466

(22) Filed: Apr. 27, 2010

(51) Int. Cl. A61M 37/00

(52) U.S. Cl. 604/65

(57) ABSTRACT

Techniques for programming therapy delivered a patient via a medical device are described. One example technique includes receiving a request for a modification to a therapy delivered to a patient via medical device, transmitting a request to a remote networking device for authorization for the modification to the therapy in response to the request for the modification to the therapy, receiving a response to the request for authorization, where the response to the request for authorization indicates whether the requested modification is authorized, and modifying the therapy according to the requested modification when the requested modification is determined to be authorized. In some examples, the medical device includes a medical fluid delivery device.

### TECHNICAL FIELD

This disclosure relates generally to medical device systems and, more particularly, to programming medical device systems.

### BACKGROUND

A variety of medical devices are used for chronic, i.e., long-term, delivery of fluid therapy to patients suffering from a variety of conditions, such as chronic pain, tremor, Parkinson's disease, epilepsy, urinary or fecal incontinence, sexual dysfunction, obesity, spasticity, or gastroparesis. For example, pumps or other fluid delivery devices can be used for chronic delivery of therapeutic agents, such as drugs, to patients. These devices are intended to provide a patient with a therapeutic output to alleviate or assist with a variety of conditions. Typically, such devices are implanted in a patient and provide a therapeutic output under specified conditions on a recurring basis.

One type of implantable fluid delivery device is a drug infusion device that can deliver a fluid medication to a patient at a selected site. A drug infusion device may be implanted at a location in the body of a patient and deliver a fluid medication through a catheter to a selected delivery site in the body. Drug infusion devices, such as implantable drug pumps, commonly include a reservoir for holding a supply of a therapeutic fluid, such as a drug, for delivery to a site in the patient. The fluid reservoir can be self-sealing and accessible through one or more ports. A pump is fluidly coupled to the reservoir for delivering the therapeutic substance to the patient. A catheter provides a pathway for delivering the therapeutic substance from the pump to the delivery site in the patient.

### SUMMARY

In general, the disclosure relates to techniques for modifying medical therapy delivered to a patient by a medical device system. In some examples, the medical device system may include a medical fluid delivery system including a fluid delivery device. While therapy is being delivered to a patient by the fluid delivery device to treat patient condition, one or more modifications to therapy may be proposed, e.g., by a patient via a patient programming device and/or as a result of a scheduled, semi-automatic therapy adjustment. Authorization may be sought by the medical fluid delivery system upon receipt of the request for therapy modification. In response to the request for authorization, the proposed modification may be authorized, e.g., by a clinician or other user. When authorization for the therapy modification is received, the medical fluid delivery system may modify the therapy delivered to the patient by the fluid delivery device according to the proposed modification.

In some examples, upon receipt of a requested therapy modification, the medical fluid delivery system may transmit a request for authorization of the proposed therapy modification to a remote device via a network to a clinician or other user. The clinician or other user may then access information regarding the requested therapy modification via the remote networking device to determine whether to authorize or deny the requested modification. The clinician or other user may then transmit information indicating whether the requested therapy modification is authorized from the remote networking device to the medical fluid delivery device via the network. Based on the transmitted authorization indication, the medical fluid delivery system may or may not implement one or more modifications to the therapy being delivered to the patient by the fluid delivery device.

In one example, the disclosure relates to a method comprising receiving a request for a modification to a therapy delivered to a patient via a medical device, transmitting a request to a remote networking device for authorization for the modification to the therapy in response to the request for the modification to the therapy, receiving a response to the request for authorization, where the response to the request for authorization indicating whether the requested modification is authorized, and modifying the therapy according to the requested modification when the requested modification is determined to be authorized.

In another example, the disclosure relates to medical therapy system comprising a medical device configured to delivery therapy to a patient; a telemetry module; and a processor, wherein the processor, using the telemetry module, is configured to receive a request for a modification to the therapy delivered to the patient via the medical device, transmit a request to a remote networking device for authorization for the modification to the therapy in response to the request for the modification to the therapy, receive a response to the request for authorization indicating whether the requested modification is authorized; and modify the therapy according to the requested modification when the requested modification is determined to be authorized.

In another example, the disclosure relates to medical therapy system comprising means for receiving a request for a modification to a therapy delivered to a patient via medical device; means for transmitting a request to a remote networking device for authorization for the modification to the therapy in response to the request for the modification to the therapy; means for receiving a response to the request for authorization, the response to the request for authorization indicating whether the requested modification is authorized; and means for modifying the therapy according to the requested modification when the requested modification is determined to be authorized.

In another example, the disclosure relates to a computer-readable storage medium comprising instructions that cause a processor to receive a request for a modification to a therapy delivered to a patient via medical device; transmit a request to a remote networking device for authorization for the modification to the therapy in response to the request for the modification to the therapy; receive a response to the request for authorization, the response to the request for authorization indicating whether the requested modification is authorized; and modify the therapy according to the requested modification when the requested modification is determined to be authorized.

The details of one or more examples disclosed herein are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example of a medical fluid delivery system including an implantable fluid delivery device configured to deliver a therapeutic fluid to a patient via a catheter.

FIG. 2 is functional block diagram illustrating an example of the implantable fluid delivery device of FIG. 1.

FIG. 3 is a functional block diagram illustrating an example of an external programmer as shown in FIG. 1.

FIG. 4 is a block diagram illustrating an example system that includes a server and one or more computing devices that are coupled to an implantable medical device and external programmer as shown in FIG. 1.

FIGS. 5-8 are flow diagrams illustrating example techniques for authorizing a requested modification to therapy delivered to a patient via an implantable fluid delivery device.

### DETAILED DESCRIPTION

In general, the disclosure relates to techniques for modifying medical therapy delivered to a patient by a medical device system. In some examples, the medical device system may include a medical fluid delivery system including a fluid delivery device. While therapy is being delivered to a patient by the fluid delivery device to treat patient condition, one or more modifications to therapy may be proposed, e.g., by a patient via a patient programming device and/or as a result of a scheduled, semi-automatic therapy adjustment. Authorization may be sought by the medical fluid delivery system upon receipt of the request for therapy modification. In response to the request for authorization, the proposed modification may be authorized, e.g., by a clinician or other user. When authorization for the therapy modification is received, the medical fluid delivery system may modify the therapy delivered to the patient by the fluid delivery device according to the proposed modification.

In some examples, upon receipt of a requested therapy modification, the medical fluid delivery system may transmit a request for authorization of the proposed therapy modification to a remote device via a network to a clinician or other user. The clinician or other user may then access information regarding the requested therapy modification via the remote networking device to determine whether to authorize or deny the requested modification. The clinician or other user may then transmit information indicating whether the requested therapy modification is authorized from the remote networking device to the medical fluid delivery system via the network. Based on the transmitted authorization indication, the medical fluid delivery system may or may not implement one or more modifications to the therapy being delivered to the patient by the fluid delivery device.

Medical devices are useful for treating, managing or otherwise controlling various patient conditions or disorders including, e.g., pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, mood disorders, or other disorders. Some medical devices, referred to herein generally as fluid delivery devices may be configured to deliver one or more fluid therapeutic agents, i.e., therapeutic fluids, alone or in combination with other therapies, such as electrical stimulation, to one or more target sites within a patient.

For example, in some cases, a fluid delivery device may deliver pain-relieving drug(s) to patients with chronic pain, insulin to a patient with diabetes, or other fluids to patients with different disorders. The device may be implanted in the patient for chronic therapy delivery (i.e., longer than a temporary, trial basis) or temporary delivery. For ease of description, examples of the disclosure are primarily described with regard to an implantable fluid delivery device for chronic therapy delivery. However, examples of the disclosure may also be applicable to external fluid delivery devices or fluid delivery device that are only partially implanted in a patient, e.g., devices including one or more percutaneously implanted catheters, for chronic or temporary therapy delivery.

Furthermore, examples of the disclosure are primarily described with regard to medical fluid delivery systems and devices for purposes of illustration. However, the disclosure is not limited to such examples. In some examples, the techniques described in this disclosure may be generally applicable to a variety of medical device systems including external and implantable medical devices (IMDs). In some examples, one or more of the techniques described in this disclosure may be applied to therapy systems including external or internal electrical stimulators such as, e.g., neurostimulators that deliver neurostimulation therapy to a patient or cardiac stimulator devices that deliver pacing, cardioversion and/or defibrillation stimulation to the heart of a patient. In one example, the techniques described herein may be applicable to implantable spinal cord stimulator (SCS) system that delivers electrical SCS, e.g., for relief of chronic pain or other symptoms. In some examples, the electrical stimulation therapy delivered to a patient by the medical device system may be used to treat tremor, Parkinson's disease, epilepsy, urinary or fecal incontinence, pelvic pain, sexual dysfunction, obesity, gastroparesis, or psychiatric disorders (e.g., depression, mania, obsessive compulsive disorder, anxiety disorders, and the like). In this manner, such a medical device system may be configured to provide therapy taking the form of deep brain stimulation (DBS), pelvic floor stimulation, gastric stimulation, or any other stimulation therapy.

An implantable fluid delivery device may deliver one or more therapeutic fluids to a patient according to one or more therapy programs. A therapy program generally refers to a program sent to an implantable fluid delivery device by a programming device (or preprogrammed on the implantable fluid delivery device) that causes the fluid delivery device to deliver fluid at certain rates and at certain times. In one example, a therapy program may include definitions for one or more of a supplemental bolus and a therapy schedule. A therapy program may include additional information, such as patient information, permissions for a user to add a supplemental bolus, as well as limits on the frequency or number of such boluses, historical therapy schedules, fluid or drug information, or other information. A therapy program may also include information regarding authorization requirements for modifications to one or more therapy programs, including therapy schedules or other therapy parameters defined for therapy delivered to a patient via the implantable fluid delivery device.

A therapy schedule generally defines the rate (which may be zero) at which to administer a fluid, or a drug or drug combination within the fluid, at specific times to a patient. In particular, the therapy schedule may define one or more programmed doses, which may be periodic or aperiodic, each dose including, e.g., a rate of fluid delivery and a time duration for which to deliver the dose. Dose generally refers to the amount of drug or other therapeutic fluid delivered over a period of time, and may change over the course of a therapy schedule such that a drug or other therapeutic fluid may be delivered at different rates at different times. Although delivery of drugs may be described for purposes of illustration, the techniques described in this disclosure may be useful in delivery of various therapeutic fluids. Accordingly, description of the delivery of drugs should not be considered limiting with respect the techniques broadly described in this disclosure.

A therapy schedule may include one or more fluid delivery profiles for determining the rate of drug delivery at a given time. A fluid delivery profile may be graphically represented by plotting the rate of fluid delivery versus time defined by a therapy schedule for a given period of time, e.g., hour, day, or week. In some examples, to maintain therapeutic efficacy therapy, the rate at which fluid is delivered to a patient can vary temporally, e.g., rather than maintaining a constant rate of fluid delivery over a given time period. Accordingly, a clinician may define one or more therapy schedules for a patient that causes the rate of a fluid delivery being delivery to the patient to change on a temporal basis. Changes to the rate at which fluid is delivered to a patient from the implantable medical device may correspond to “steps” of a fluid delivery profile.

In some examples, one or more therapy profiles may be defined by a clinician for weaning a patient off a drug or from a higher dosage to a lower dosage. In most cases, such a therapy profile includes a series of step reductions that serve to generally reduce the rate of fluid delivery to the patient over a suitable time period. In this manner, one or more undesired side-effects for a patient associated with a reduced drug delivery (e.g., side-effects from drug withdrawal) may be lessened or avoided by weaning the patient from the drug rather than drastically reducing the delivery of a drug over a short period of time or simply stopping the delivery of the drug to the patient at single point in time.

Similarly, one or more therapy profile may be defined by a clinician for ramping-up a patient from a lower dosage to higher target dosages. In most cases, such a therapy profile includes a series of step increases that serve to generally increase the rate of fluid delivery to the patient over a suitable time period. In this manner, one or more undesired side effects for a patient associated with the increased drug delivery may be lessened or avoided by incrementally increasing the drug delivered to the patient over a suitable period of time rather than drastically increasing the delivery of drug over a short period of time.

In most cases, a clinician creates the one or more therapy programs that a fluid delivery device will use to deliver therapy to a patient during an initial programming session. In the case of implantable fluid delivery device, the initial programming session may occur shortly after the device is implanted in the patient. The values for each of the parameters of a program, e.g., a fluid delivery rate and/or time period defined by a therapy profile, may have a significant impact on the efficacy and side effects of the delivery of therapy according to that therapy program. The process of selecting values for the therapy parameters that provide adequate results can be time consuming.

Even after this often-lengthy process, the therapy programs selected during an initial programming session may ultimately prove to be inadequate. The eventual inadequacy of the initial programming may be due to a variety of problems including, but not limited to, progression of symptoms and/or an underlying ailment, increased or changed symptoms or side effects during activities and/or postures that were not replicated in the clinic during the initial programming session, and slow onset of side effects. In some cases, the therapy programs selected during an initial programming session may prove to be inadequate, and the patient must return to the clinic for a follow-up programming session. Multiple follow-up programming sessions may be required over the period of time that the fluid delivery device is used to deliver therapy to the patient.

Such programming techniques may be time consuming and inconvenient for both a patient and clinician. In limited situations, a patient may be allowed to make one or more modifications to a therapy program outside of a programming session. However, because certain therapy modifications, such as, e.g., adjustments to rate of fluid delivery of a therapy profile, may cause the patient to experience one or more undesired side effects, the ability of a patient to independently modify a therapy program outside the supervision of a clinician may be limited to relatively minor adjustment or modifications to therapy parameters not capable of resulting in undesired patient side effects.

As will be described in further detail below, one or more examples of the disclosure relate to medical fluid delivery systems including fluid delivery device(s) for delivery of medical therapy to a patient in which proposed therapy modifications are authorized prior to being implemented by the medical fluid delivery system but after the therapy modification has been requested, e.g., by a patient or based on a semi-automatic scheduled modification. The medical therapy system may be configured to request authorization for all proposed therapy modifications or for only those therapy modifications defined, e.g., by a clinician during a programming session, as requiring authorization before being implemented by the medical device system. The requested therapy modification may be remotely reviewed by a clinician via a remote networking device communicatively coupled to the fluid delivery device.

When a proposed request for a therapy modification requiring authorization is received, the medical device system may transmit a request to an external device, e.g., to a remote networking device, for authorization of the proposed therapy modification. In some examples, the request is initiated by the implanted fluid delivery device or by an external patient programming device, and then transmitted to a remote networking device for review by a remote user. A user such as a clinician may review the requested therapy modification, and respond by transmitting an indication as to whether or not the proposed therapy modification should be implemented by the fluid delivery device. The implanted fluid delivery device may implement or reject the proposed therapy modification based on whether the response indicates that the proposed modification is authorized or not by the clinician.

For ease of illustration, examples of the disclosure are primarily described with regard to modifications to a therapy program, such as modifications to one or more parameters of a therapy profile (e.g., adjustments to the rate of fluid delivery) corresponding to a therapy schedule. However, examples of the disclosure are not limited to such therapy modifications but instead may be applied to the modification of any parameters. For example, therapy modifications may include, but are not limited to: changes in the maximum number of patient requested boluses, e.g., within a time window that can be serviced; changes to the rate of change (e.g., slope of ramp up and/or ramp down) within a programmed sequence; changes to the rate of the programmed endpoints of a ramping up or ramping down sequence; a change in a therapy schedule to match user activity or address symptomatic conditions; a shift in time of the therapy, e.g., to correspond to a relocation to a different time zone; adjustment of the maximum bolus delivery rate, e.g., based on environmental conditions (pressure) or battery level of the device; activity based therapy response adjustments; the adjustment of flow through multiple fluid paths in multi-site therapy delivery; the application of a default delivery rate in response to the detection of component degradation within a fluid delivery device; and a change in the configuration of device alarms to make the alarms more sensitive, less sensitive, or to disable one or more alarms entirely, e.g., for cases where a patient is experiencing an undesirable level of false device alarms. Other therapy modifications are contemplated.

Modification to a therapy profile may include adjustments to the rate at which a fluid is delivered to a patient and/or adjusting the time period over which the fluid is being delivered to the patient at the rate defined by the therapy profile. In some examples, such modification may be accomplished by redefining the value of one or more parameters from an existing therapy profile, e.g., delivery rate and/or time period values for a given therapy profile, or may include switching from one therapy profile to another programmed therapy profile available for defining the therapy delivered by the fluid delivery device. In some examples, a therapy modification may in essence generate a new therapy profile defining the rate at which a fluid is delivered to a patient via the implanted fluid delivery device on a temporal basis.

A therapy modification may be proposed by a patient outside of a clinician supervised programming session. In some examples, a patient may propose one or more modifications to a therapy program via a patient programming device. For example, using an external patient programmer, a patient may request an increase or decrease in the rate at which the fluid is being delivered to the patient by the implanted fluid delivery device. A patient may request the therapy modification based on the therapeutic efficacy resulting from the one or more therapy profiles defining the therapy being delivered to the patient at that time. If authorized, a proportional rate increase or decrease may be globally applied to all the rates defined by a therapy profile or may be applied only to the delivery rate defined for the time period that the rate increased or decrease was requested by the patient.

Alternatively or additionally, a therapy modification may correspond to a scheduled modification to a therapy profile which requires authorization before the modification is implemented by the fluid delivery device. In some examples, a clinician may define a therapy profile in which therapy modifications, e.g., changes to fluid delivery rate, are scheduled but require authorization before implementing the proposed therapy modification. For example, in the case of a therapy profile designed to wean a patient from a drug delivered by the fluid delivery systems, periodic reductions in the rate at which a fluid is delivered to a patient may be scheduled. However, the scheduled modification may be semi-automatic in the sense that a clinician must first authorize the modification, e.g., based on the patient condition at the time of the schedule modification, before the modification is actually made to the therapy profile, e.g., by further reducing the delivery rate to the scheduled value. In such a case, a request for the therapy modification may be transmitted to a clinician or other user, and the clinician or other user may review the request to determine whether or not the scheduled therapy modification is desirable. Alternatively or additionally, a scheduled therapy modification may be triggered by the satisfaction of a predefined patient condition, e.g., a condition related to patient pain score, activity level, heart rate, weight change, and the like. In some examples, a clinician or other user may evaluate the requested therapy modification in view of related supplemental authorization information including patient physiological condition information to determine whether or not to authorize the schedule therapy modification. Such supplemental authorization may be stored in the fluid delivery device, an external programming device, and/or remotely with a clinician by a remote networking device.

In the case of remote authorization, proposed therapy modifications may be transmitted from the fluid delivery device and/or patient programming device to a clinician or other user at a remote location via a network. The clinician or other user may access information regarding the proposed modification via a remote networking device. In some examples, the request for authorization may include an alert or notification that a therapy modification has been proposed, in which case the clinician or other user may use the remote networking device to access information regarding the proposed modification as well as supplemental authorization information to the extent the user requires. Based on a review of the proposed therapy modification using the remote networking device, the clinician or other user may then transmit an indication to the fluid delivery device via the network indicating whether or not the proposed therapy modification is authorized. The fluid delivery device may implement or deny a therapy modification in accordance with the indication provided by the clinician or other user. In some examples, the modification authorized by a user may be different than that requested by the patient or denied altogether, e.g., as deemed appropriate by the user in view of one or more changes in the operational status and/or patient condition which has occurred between the time of request and subsequent authorization. In this manner, one or more therapy modifications may be under the supervision of a clinician without requiring a patient and clinician to convene during an in-clinic programming session.

Requests for authorization for therapy modifications may be transmitted to a clinician or other user in or near real time with the receipt of the proposed therapy modification. In other examples, proposed therapy modifications may be transmitted on a periodic basis, e.g., corresponding to the timing of periodic interrogation of an implanted fluid delivery device by an external monitoring device such as a bedside monitor. A clinician or other user may review proposed modification substantially immediately upon receipt of the request for authorization, e.g., in cases in which a proposed modification is relatively urgent, while in other examples, a clinician or other user may periodically review one or more therapy modification requests that have been received since the clinicians or other users last request review, e.g., on a daily, weekly, or monthly basis.

A medical device system may be programmed by a user such as a clinician to characterize the level and/or type of authorization required for particular therapy modifications. Such characterization by a clinician may occur during an initial programming session or other programming. In some examples, a user may program an implantable fluid delivery device to require that certain therapy modifications require authorization prior to implementation of the therapy modifications, while other therapy modifications may be programmed as not requiring authorization after being requested, e.g., by a patient, before being implemented. In such examples, possible therapy modification may be considered either preauthorized therapy modification or therapy modifications that require authorization after the therapy modification is requested before being implemented.

In some examples, a clinician or other user may access supplemental authorization information for patient via a remote networking device to assist the user in determining whether or not to authorize a proposed therapy modification. Supplemental authorization information may include patient physiological condition information (e.g., blood pressure, heart rate, body temperature, blood glucose level, weight, patient pain score, and the like) that may be relevant in deciding whether to authorize a proposed therapy modification. Additionally or alternatively, supplemental authorization information may include information relating to the operational integrity of the fluid delivery device (e.g., information whether the fluid delivery device is properly delivering fluid to the patient according to one or more therapy programs) that may again be relevant in deciding whether to authorize a proposed therapy modification. A medical device system may be configured to transmit supplemental authorization information along with a request for authorization and/or a user may request that supplemental authorization information by provided by the medical device system after reviewing a proposed therapy modification.

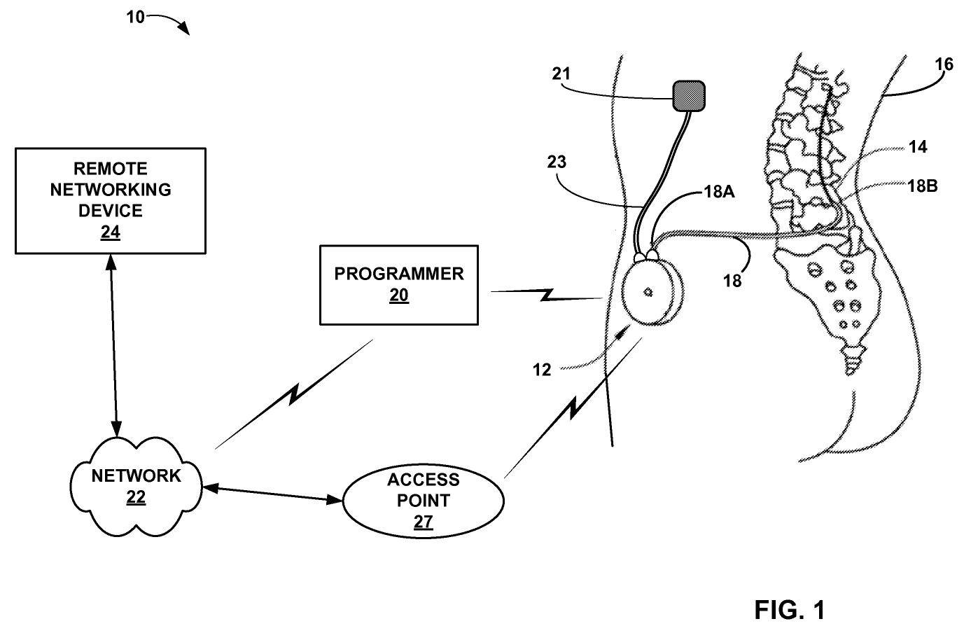

FIG. 1 is a conceptual diagram illustrating an example of a medical therapy system 10 for delivering a therapeutic fluid to patient 16. Therapy system 10 includes implantable medical device (IMD) 12, catheter 18, external programmer 20, sensor 21, network 22, and remote networking device 24. IMD 12, external programmer 20, and/or sensor 21 may directly or indirectly communicate with remote networking device 24 via network 22. Therapy system 10 may, in other examples, include additional components. For example, instead of being incorporated into IMD 12 as described below, therapy system 10 may include an external or separately implanted telemetry module for communication between IMD 12, programmer 20, and/or sensor 21.

Therapy system 10 delivers a medical therapy to patient 16. In the example of FIG. 1, IMD 12 is connected to catheter 18 to deliver at least one therapeutic fluid agent, such as a pharmaceutical agent, pain relieving agent, anti-inflammatory agent, gene therapy agent, or the like, to a target site within patient 16. Example therapeutic agents that IMD 12 can be configured to deliver include, but are not limited to, insulin, morphine, hydromorphone, bupivacaine, clonidine, other analgesics, baclofen and other muscle relaxers and antispastic agents, genetic agents, antibiotics, nutritional fluids, hormones or hormonal drugs, gene therapy drugs, anticoagulants, cardiovascular medications or chemotherapeutics.

IMD 12 may include a reservoir, pump and controller for delivery of a therapeutic fluid via catheter 18. IMD 12 and catheter 18 together form an implantable fluid delivery device. Additionally or alternatively, IMD 12 may be configured to deliver a therapeutic fluid to patient 16 without catheter 18, e.g., via one or more delivery ports formed in the housing of IMD 12. In the example of FIG. 1, the fluid delivery device is fully implantable within the patient, but may communicate with external devices such as programmer 20 or network 22 via wireless telemetry, and receive refill of therapeutic fluid via percutaneous injection. In other examples, the fluid delivery device may be partially implantable. For example, the reservoir, pump and controller may be external to the patient, while catheter 18 may be implantable within the patient, and coupled to the external pump via a percutaneous port. Hence, the techniques described in this disclosure may be especially useful with fully implantable fluid delivery device including implantable reservoir, pump, controller and catheter, but also may be used with a partially implantable fluid delivery device or external fluid delivery device.

In the example of FIG. 1, the therapeutic agent is a therapeutic fluid, which IMD 12 delivers to patient 16 through catheter 18 from a proximal end 18A coupled to IMD 12 to distal end 18B located proximate to the target site. Catheter 18 can comprise a unitary catheter or a plurality of catheter segments connected together to form an overall catheter length. External programmer 20 is configured to wirelessly communicate with IMD 12 as needed, such as to provide or retrieve therapy information or control aspects of therapy delivery (e.g., modify the therapy parameters such as rate or timing of delivery, turn IMD 12 on or off, and so forth) from IMD 12 to patient 16. In some examples, patient 16 may request a modification to the therapy delivered from the implantable fluid delivery device via external programmer 20, e.g., by requesting one or more adjustments to therapy parameter values define by a therapy profile. If the requested modification requires authorization prior to being implemented, external programmer 20 may communicate with remote networking device 24 via network 22 to receive authorization for the therapy modification, e.g., from a clinician or other user, prior to implementing the requested modification.

IMD 12 may have an outer housing that is constructed of a biocompatible material that resists corrosion and degradation from bodily fluids including, e.g., titanium or biologically inert polymers. IMD 12 may be implanted within a subcutaneous pocket relatively close to the therapy delivery site. For example, in the example shown in FIG. 1, IMD 12 is implanted within an abdomen of patient 16. In other examples, IMD 12 may be implanted within other suitable sites within patient 16, which may depend, for example, on the target site within patient 16 for the delivery of the therapeutic agent. In still other examples, as discussed above, instead of providing fully implantable IMD 12, one or more components of IMD 12 may be external to patient 16 with a percutaneous catheter connected between such components and the target delivery site within patient 16, providing an at least partially implantable fluid delivery device. In general, however, fully implantable fluid delivery devices are described in this disclosure for purposes of illustration.

Catheter 18 may be coupled to IMD 12 either directly or with the aid of a catheter extension (not shown in FIG. 1). In the example shown in FIG. 1, catheter 18 traverses from the implant site of IMD 12 to one or more targets proximate to spine 14. Catheter 18 is positioned such that one or more fluid delivery outlets (not shown in FIG. 1) of catheter 18 are proximate to the targets within patient 16. In the example of FIG. 1, IMD 12 delivers a therapeutic agent through catheter 18 to targets proximate to spinal cord 14. IMD 12 can be configured for intrathecal drug delivery into the intrathecal space, as well as epidural delivery into the epidural space, both of which surround spinal cord 14. The epidural space (also known as “extradural space” or “peridural space”) is the space within the spinal canal (formed by the surrounding vertebrae) lying outside the dura mater, which encloses the arachnoid mater, subarachnoid space, the cerebrospinal fluid, and spinal cord 14. The intrathecal space is within the subarachnoid space, which is further inward past the epidural space and dura mater and through the theca.

Although the target site shown in FIG. 1 is proximate to spinal cord 14 of patient 16, other applications of therapy system 10 include alternative target delivery sites. The target delivery site in other applications of therapy system 10 can be located within patient 16 proximate to, e.g., sacral nerves (e.g., the S2, S3, or S4 sacral nerves) or any other suitable nerve, organ, muscle or muscle group in patient 16, which may be selected based on, for example, a patient condition. In one such application, therapy system 10 may be used to deliver a therapeutic agent to tissue proximate to a pudendal nerve, a perineal nerve or other areas of the nervous system, in which cases, catheter 18 would be implanted and substantially fixed proximate to the respective nerve. Positioning catheter 18 to deliver a therapeutic fluid agent to various sites within patient 16 enables therapy system 10 to assist in managing, e.g., peripheral neuropathy or post-operative pain mitigation, ilioinguinal nerve therapy, intercostal nerve therapy, gastric drug induced stimulation for the treatment of gastric motility disorders and/or obesity, and muscle stimulation, or for mitigation of other peripheral and localized pain (e.g., leg pain or back pain).

As another example delivery site, catheter 18 may be positioned to deliver a therapeutic agent to a deep brain site or within the heart (e.g., intraventricular delivery of the agent) or blood vessels. Delivery of a therapeutic agent within the brain may help manage any number of disorders or diseases including, e.g., chronic pain, diabetes, depression or other mood disorders, dementia, obsessive-compulsive disorder, migraines, obesity, and movement disorders, such as Parkinson's disease, spasticity, and epilepsy. Catheter 18 may also be positioned to deliver insulin to a patient with diabetes. Although IMD 12 is shown implanted in the lower back of patient 16, it shall be understood that IMD 12 may be implanted at any suitable location within patient 16, including, but not limited to, the cranium, abdomen, the chest, or buttocks of patient 16.

In some examples, multiple catheters 18 may be coupled to IMD 12 to target the same or different tissue or nerve sites within patient 16. Thus, although a single catheter 18 is shown in FIG. 1, in other examples, system 10 may include multiple catheters or catheter 18 may define multiple lumens for delivering different therapeutic agents to patient 16 or for delivering a therapeutic agent to different tissue sites within patient 16. Accordingly, in some examples, IMD 12 may include a plurality of reservoirs for storing more than one type of therapeutic agent. In some examples, IMD 12 may include a single long tube that contains the therapeutic agent in place of a reservoir. However, for ease of description, an IMD 12 including a single reservoir is primarily discussed in this disclosure with reference to the example of FIG. 1.

Sensor 21 may be used to monitor information related to the delivery of therapy to patient 16. In some examples, sensor 21 may be configured to collect information regarding one or both of the efficacy of therapy being delivered by IMD 12, or side effects resulting from the therapy. Information collected by sensor 21 may be used by therapy system 10 to identify the need to initiate, terminate, and/or adjust the delivery of therapy to patient 16 from IMD 12.

In some examples, sensor 21 may be any sensor that senses or responds to any physiological parameter of patient 16 associated with the therapy provided by IMD 12. For example, sensor 21 may be a mechanical sensor, an electrical sensor, a chemical sensor, and/or an electrochemical sensor. In some examples, sensor 21 may be a scale, a blood glucose monitor, an accelerometer, a moisture sensor, or may measure temperature, auditory levels, and the like. In some embodiments, the sensor 21 may measure, for example, activity levels of a patient 16, glucose levels, impedance, distension of the stomach or other organs, urine flow, urine or blood pH, body temperature, bladder contraction, brain electrical activity, electroencephalogram (EEG) morphology, pulse rate, respiration rate, patient weight, and the like. While sensor 21 is illustrated as an implantable sensor, sensor 21 may additionally or alternatively be an external sensor, e.g., an external sensor that may be worn or otherwise physically coupled to patient 16 and/or an external sensor incorporated into programmer 20. Sensor 21 may be a separate component from that of IMD 12 and/or catheter 18, or may be incorporated into IMD 12 and/or catheter 18 (e.g., located internally within IMD 12). In other examples, system 10 may not include sensor 21.

In some examples, sensor 21 may collect information relating to the operation or performance of IMD 12 and catheter 18 in delivering the therapeutic fluid to patient 16. For example, information sensed by sensor 21 may be used to identify the occurrence of one or more occlusions or fractures in catheter 18 that may prevent or inhibit delivery of the therapeutic fluid from IMD 12 to the target tissue site of patient 16. As another example, sensor 21 may collect information used to identify the migration of catheter 18 within patient 16 that may cause fluid to be delivered to a tissue site not targeted for the delivery of therapy. Alternatively or additionally, the sensor information may relate to self tests performed by IMD 12 to monitor the integrity of the memory, programming data, and/or individual components used to deliver fluid to patient 16. In such examples, the one or more parameters monitored by sensor 21 may be indicative of the operational integrity of IMD 12 and/or catheter 18 with regard to the ability of the device to deliver therapy to patient 18 as intended. In some examples, operational integrity may be monitored based on occurrences of low or empty reservoir detection or periodic pump stall/recovery detection, the presence of one or more catheter leaks along the delivery path, and/or the efficiency of the fluid delivery, e.g., based on battery life of IMD 12. Example parameters monitored by sensor 21 may include the decay rate of pump pulse catheter pressurization waves, the magnitude of sensed physiological signal amplitudes, such as, e.g., cardiac and respiratory pressure, and/or the base pressure of Cerebral Spinal Fluid.

Data collected by sensor 21 may be communicated to IMD 12, programmer 16, and/or remote networking device 24. In some examples, IMD 12 may include or be communicatively coupled to sensor 21. In the example shown in FIG. 1, IMD 12 is coupled to sensor 21 via lead 23. In other examples, sensor 21 may communicate wirelessly with IMD 12 and/or programmer 20 to transmit sensing data. In some examples, sensor 21 may communicate directly with programmer 20, or more communicate indirectly with programmer 20 via IMD 12. Sensor 21 may directly communicate the sensor data over network 22 to remote networking device 24. In other embodiments, sensor 21 may communicate the sensor data to one or both of IMD 12 or programmer 16, which may in turn communicate the sensor data over network 22 to remote networking device 24.

As will be described further below, in some examples, sensor 21 may be used to collect supplemental authorization information which may be used by a clinician or other user to determine whether a requested therapy modification should be authorized. In some examples, the supplemental authorization information includes patient physiological condition information regarding the efficacy of therapy being administered by IMD 12 or general information regarding the physiological condition of patient 16 sensed via sensor 21. Patient physiological condition information may include sensed physiological parameter information for patient 16, such as, e.g., one or more of blood pressure value, a blood glucose level, body temperature, patient activity, patient spasticity, or patient respiration. In some examples, supplemental authorization information may include patient pain score information, e.g., paint score information generated based at least in part on patient pain feedback received via programmer 20. Additionally or alternatively, the supplemental authorization information may be indicative of the operational integrity of IMD 12 and/or catheter 18 with regard to the ability of IMD 12 and/or catheter 18 to deliver therapy to patient 16 as desired. Such supplemental authorization information may be accessed by a clinician or other user, e.g., via remote networking device 24, to assist the user in determining whether or not to authorize a request therapy modification transmitted to the user for approval via network 22.

Programmer 20 is an external computing device that is configured to communicate with IMD 12 via wireless or wired telemetry. In some examples, programmer 20 may be a hand-held computing device that includes a display viewable by the user and a user input mechanism that can be used to provide input to programmer 20. For example, programmer 20 may include a display screen (e.g., a liquid crystal display or a light emitting diode display) that presents information to the user. In addition, programmer 20 may include a keypad, buttons, a peripheral pointing device, touch screen, voice recognition, or another input mechanism that allows the user to navigate though the user interface of programmer 20 and provide input.

In one example, the display may allow a patient to monitor his or her therapy. In some embodiments, an input device may allow the patient to initiate therapy, request changes to one or more parameters of one or more therapy programs, and/or input information regarding the effects of therapy, e.g., to generate pain score information. In some embodiments, a clinician may utilize a programmer 20 to interrogate IMD 12 or make changes to the therapy parameter settings. The clinician programmer may include additional or alternative programming features relative to the patient programmer. For example, more complex or sensitive tasks may only be allowed by the clinician programmer to prevent patient 16 from making undesired or unsafe changes to the operation of IMD 12.

If programmer 20 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 20 may be a touch screen that allows the user to provide input directly to the user interface shown on the display. The user may use a stylus or his/her finger to provide input to the display.

In other examples, rather than being a handheld computing device or a dedicated computing device, programmer 20 may be a larger workstation or a separate application within another multi-function device. For example, the multi-function device may be a cellular phone, personal computer, laptop, workstation computer, or personal digital assistant that can be configured with an application to simulate programmer 20. Alternatively, a notebook computer, tablet computer, or other personal computer may enter an application to become programmer 20 with a wireless adapter connected to the personal computer for communicating with IMD 12.

When programmer 20 is configured for use by the clinician, programmer 20 may be used to transmit initial programming information to IMD 12. This initial information may include hardware information for system 10 such as the type of catheter 18, the position of catheter 18 within patient 16, the type and amount, e.g., by volume of therapeutic agent(s) delivered by IMD 12, a refill interval for the therapeutic agent(s), a baseline orientation of at least a portion of IMD 12 relative to a reference point, therapy parameters of therapy programs stored within IMD 12 or within programmer 20, and any other information the clinician desires to program into IMD 12.

The clinician uses programmer 20 to program IMD 12 with one or more therapy programs that define the therapy delivered by the IMD. During a programming session, the clinician may define one or more therapy programs that may provide effective therapy to patient 16. Patient 16 may provide feedback to the clinician as to efficacy of a program being evaluated or desired modifications to the program. Once the clinician has identified one or more programs that may be beneficial to patient 16, the patient may continue the evaluation process and determine which therapy program best alleviates the condition of the patient or otherwise provides efficacious therapy to the patient.

In some cases, programmer 20 may also be configured for use by patient 16. When configured as the patient programmer, programmer 20 may have limited functionality in order to prevent patient 16 from altering critical functions or applications that may be detrimental to patient 16. In this manner, programmer 20 may only allow patient 16 to adjust certain therapy parameters or set an available range for a particular therapy parameter. In some cases, a patient programmer may permit the patient to control IMD 12 to deliver a supplemental, patient bolus, if permitted by the applicable therapy program administered by the IMD, e.g., if delivery of a patient bolus would not violate a lockout interval or maximum dosage limit. Using programmer 20, patient 12 may request one or more therapy modifications described in the disclosure which may require authorization after being requested to be implemented by IMD 12. Programmer 20 may also provide an indication to patient 16 when therapy is being delivered or when IMD 12 needs to be refilled or when the power source within programmer 20 or IMD 12 need to be replaced or recharged.

Whether programmer 20 is configured for clinician or patient use, programmer 20 may communicate to IMD 12, sensor 21 or any other computing device via wireless or wired communication. Programmer 20, for example, may communicate via wireless communication with IMD 12 using radio frequency (RF) telemetry techniques. Programmer 20 may also communicate with another programmer or computing device via a wired or wireless connection using any of a variety of communication techniques including, e.g., RF communication according to the 802.11 or Bluetooth specification sets, infrared (IR) communication according to the IRDA specification set, or other standard or proprietary telemetry protocols. Programmer 20 may also communicate with another programming or computing device via exchange of removable media, such as magnetic or optical disks, or memory cards or sticks including, e.g., non-volatile memory. Further, programmer 20 may communicate with IMD 12 and another programmer via, e.g., a local area network (LAN), wide area network (WAN), public switched telephone network (PSTN), or cellular telephone network, or any other terrestrial or satellite network appropriate for use with programmer 20 and IMD 12.

Communication between IMD 12, sensor 21, programmer 20, remote networking device 24, and other therapy system components may allow two-way transfer of information between components of therapy system 10. In one example, programmer 20 transfers control data, such as one or more therapy programs, to IMD 12. Similarly, remote networking device 24 may communicate control data, such as one or more therapy programs, to IMD 12 via network 22 to allow a clinician or other user to remotely program IMD 12. In some embodiments, IMD 12 may transmit patient physiological condition information relating to the therapy delivered by IMD 12 or other information to programmer 20 and/or remote networking device 24. As one example, physiological conditions information communicated from IMD 12 may include physiological parameter information or other supplemental authorization information collected via sensor 21.

Network 22 may facilitate communication between programmer 20, IMD 12, sensor 21 and remote networking device 24. In this way, data may be exchanged between two or more of the apparatuses. Network 22 may, as examples, include one or more of a local area network (LAN), wide area network (WAN), public switched telephone network (PSTN), or cellular telephone network. Remote networking device 24 may allow a clinician or another authorized user, such as a technical expert, to communicate remotely with IMD 12 or other components of system 10 via network 22. Remote networking device 24 may be any computing device with the ability to contact a network, such as a cellular telephone, personal digital assistant (PDA), tablet personal computer (PC), laptop, desktop PC, workstation, or the like. Using remote networking device 24, a clinician may access data collected by programmer 20, IMD 12, sensor 21, or information entered by patient 16. In some examples, based on an analysis of collected data by a clinician using remote networking device, a clinician (or other user) may determine whether or not to authorize a requested modification to therapy delivered to patient 16 via IMD 12, and communicate the determination to IMD 12 or programmer 20 via network 22.

IMD 12 or other component of system 10 may utilize access point 27 to communicate with remote networking device 24 via network 22. Access point 27 may comprise a device, such as a home monitoring device, that connects to network 22 via any of a variety of connections, such as telephone dial-up, digital subscriber line (DSL), or cable modem connections. In other embodiments, access point 27 may be coupled to network 22 through different forms of connections, including wired or wireless connections.

As described above, in some applications, therapy system 10 can be used to reduce pain experienced by patient 16 or otherwise manage a patient condition via delivery of therapy. In such an application, IMD 12 can deliver one or more therapeutic agents to patient 16 according to one or more therapy programs that set forth different therapy parameters, such as a therapy schedule defining delivery rates for the programmed doses, and specific times to deliver the programmed doses. The therapy programs may be a part of a program group for therapy, where the group includes a plurality of therapy programs. In some examples, IMD 12 may be configured to deliver a therapeutic agent to patient 16 according to different therapy schedules on a selective basis. IMD 12 may include a memory to store one or more therapy programs, instructions defining the extent to which patient 16 may adjust therapy parameters (which may or may not be subject to post-request authorization), switch between therapy programs, or undertake other therapy modifications. Patient 16 or a clinician may select and/or generate additional therapy programs for use by IMD 12 via external programmer 20 and/or remote networking device at any time during therapy or as designated by the clinician.

The therapy program information may set forth therapy parameters, such as different predetermined dosages of the therapeutic agent (e.g., a dose amount), the rate of delivery of the therapeutic agent (e.g., rate of delivery of the fluid), the maximum acceptable dose, a time interval between successive supplemental boluses such as patient-initiated boluses (e.g., a lock-out interval), a maximum dose that may be delivered over a given time interval, and so forth. IMD 12 may include a feature that prevents dosing the therapeutic agent in a manner inconsistent with the therapy program. Programmer 20 may assist the clinician in the creation/identification of dosing programs by providing a methodical system of identifying potentially beneficial therapy parameters.

A dosage of a therapeutic agent, such as a drug, may be expressed as an amount of drug, e.g., measured in milligrams or other volumetric units, provided to patient 16 over a time interval, e.g., per day or twenty-four hour period. In this sense, the dosage may indicate a rate of delivery. This dosage amount may convey to the caregiver an indication of the probable efficacy of the drug and the possibility of side effects. In general, a sufficient amount of the drug should be administered in order to have a desired therapeutic effect, such as pain relief. However, the amount of the drug administered to the patient should be limited to a maximum amount, such as a maximum daily dose, in order to avoid potential side effects. Program information specified by a user via programmer 20 may be used to control dosage amount, dosage rate, dosage time, maximum dose for a given time interval (e.g., daily), or other parameters associated with delivery of a drug or other fluid by IMD 12. Dosage may also prescribe particular concentrations of active ingredients in the therapeutic agent delivered by IMD 12 to patient 16.

In accordance with some examples of the disclosure, system 10 may be configured to receive one or more requested therapy modifications, e.g., from patient 16 via programmer 20 and/or scheduled therapy modifications from programmer 20 or IMD 12, and receive approval for the requested therapy modification from a remote user such as clinician. Programmer 20 and/or IMD 12 may communicate a request for authorization to remote networking device 24 via network 22. A clinician or other user may review the requested therapy modification and indicate whether or not the requested therapy modification is authorized or denied. An indication of the user's authorization decision may be transmitted from remote networking device 24 to programmer 20 and/or IMD 12 via network 22. Programmer 20 and/or IMD 12 may implement the proposed therapy modification in cases in which the clinician indicates that the requested therapy modification is authorized, while the therapy modification may not be implemented in cases in which the clinician does not authorize the requested modification (e.g., where the clinician affirmatively denies the modification or does not respond to the request for modification). In this manner, system 10 may allow for the modification of the therapy delivered to patient 16 from IMD 12.

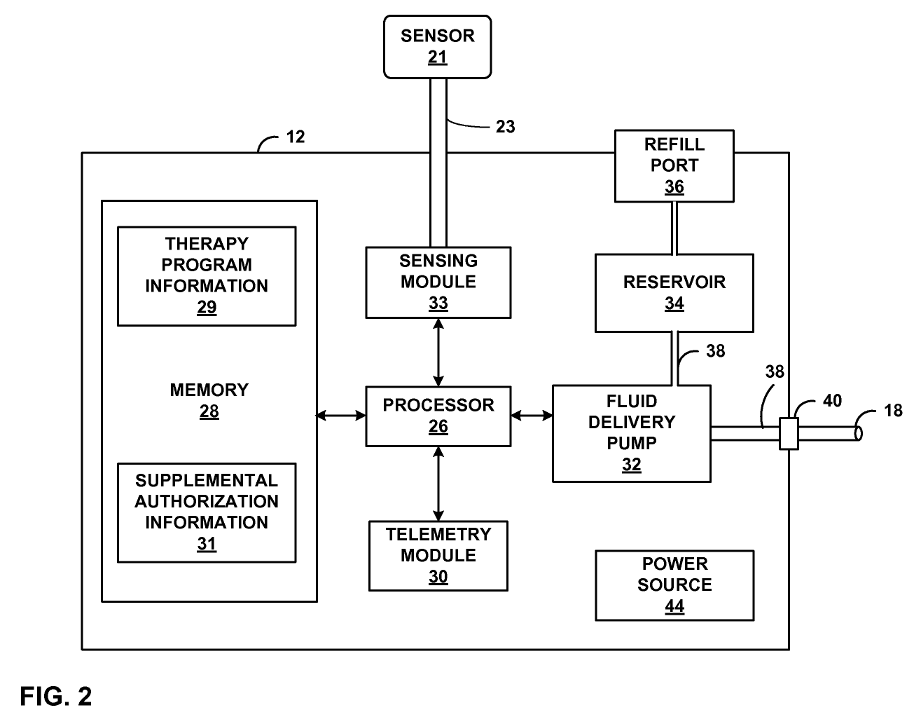

FIG. 2 is a functional block diagram illustrating components of an example of IMD 12, which includes processor 26, memory 28, telemetry module 30, fluid delivery pump 32, sensing module 33, reservoir 34, refill port 36, internal tubing 38, catheter access port 40, and power source 44. Processor 26 is communicatively connected to memory 28, telemetry module 30, sensing module 33, and fluid delivery pump 32. Fluid delivery pump 32 is connected to reservoir 34 and internal tubing 38. Reservoir 34 is connected to refill port 36. Catheter access port 40 is connected to internal tubing 38 and catheter 18. IMD 12 also includes power source 44, which is configured to deliver operating power to various components of the IMD.

During operation of IMD 12, processor 26 operates as a controller that controls fluid delivery pump 32 with the aid of instructions associated with therapy program information 29 that is stored in memory 28 to deliver a therapeutic agent to patient 16 via catheter 18. Instructions executed by processor 26 may, for example, be defined by one or more therapy programs that specify the amount of a therapeutic agent that is delivered to a target tissue site within patient 16 from reservoir 30 via catheter 18. The instructions may further specify the time at which the agent will be delivered and the time interval over which the agent will be delivered. The amount of the agent and the time over which the agent will be delivered are a function of, or alternatively determine, the rate at which the fluid is delivered to patient 16. The therapy programs may also include other therapy parameters, such as the frequency of bolus delivery, the type of therapeutic agent delivered if IMD 12 is configured to deliver more than one type of therapeutic agent, and so forth. Components described as processors within IMD 12, external programmer 20, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination.

Upon instruction from processor 26, fluid delivery pump 32 draws fluid from reservoir 34 and pumps the fluid through internal tubing 38 to catheter 18 through which the fluid is delivered to patient 16 to effect one or more of the treatments described above. Internal tubing 38 is a segment of tubing or a series of cavities within IMD 12 that run from reservoir 34, around or through fluid delivery pump 32 to catheter access port 40. Fluid delivery pump 32 can be any mechanism that delivers a therapeutic agent in some metered or other desired flow dosage to the therapy site within patient 16 from reservoir 30 via implanted catheter 18.

In one example, fluid delivery pump 32 can be a squeeze pump that squeezes internal tubing 38 in a controlled manner, e.g., such as a peristaltic pump, to progressively move fluid from reservoir 34 to the distal end of catheter 18 and then into patient 16 according to parameters specified by a set of program information stored on memory 28 and executed by processor 26. Fluid delivery pump 32 can also be an axial pump, a centrifugal pump, a pusher plate, a piston-driven pump, or other means for moving fluid through internal tubing 38 and catheter 18. In one particular example, fluid delivery pump 32 can be an electromechanical pump that delivers fluid by the application of pressure generated by a piston that moves in the presence of a varying magnetic field and that is configured to draw fluid from reservoir 34 and pump the fluid through internal tubing 38 and catheter 18 to patient 16.

Periodically, fluid may need to be supplied percutaneously to reservoir 34 because all of a therapeutic agent has been or will be delivered to patient 16, or because a clinician wishes to replace an existing agent with a different agent or similar agent with different concentrations of therapeutic ingredients. Refill port 26 can therefore comprise a self-sealing membrane to prevent loss of therapeutic agent delivered to reservoir 30 via refill port 26. For example, after a percutaneous delivery system, e.g., a hypodermic needle, penetrates the membrane of refill port 26, the membrane may seal shut when the needle is removed from refill port 26. The time period between which a therapeutic fluid is refilled into reservoir 30 of IMD 12 via refill port 26, i.e. the refill interval for the fluid, may be based on one or both of the amount of fluid volume delivered to patient 16 by IMD 12 and the expiration time of the fluid.

Sensing module 33 monitors signals generated by sensor 21 in order to monitor one or more physiological parameters of patient 16 or other system parameters sensed via sensor 21. The sensed information may be utilized by processor 26 to evaluate the therapeutic efficacy of the therapy delivered to patient 16. For example, sensing module 33 may be configured to monitor one or more of patient temperature, heart rate, blood sugar concentration, and the like, which may be indicators of therapeutic efficacy of the therapy being delivered to patient 16. In some examples, the information collected by sensing module 33 via sensor 21 may be utilized by processor to determine lock-out periods for therapy delivery. Additionally or alternatively, sensing module 33 may be configured to monitor the operational condition of IMD 12 and/or catheter 18 via sensor 21. For example, sensing module 33 may utilized sensed information to identify the presence of one or more occlusion or fractures in catheter 18 that may interfere with the delivery of therapeutic fluid from IMD 12 to patient 16. The operational status of IMD 12 may be communicate to programmer 20 and/or remote networking device for further evaluation by a user such as a clinician or patient 16.

Memory 28 of IMD 12 may store therapy program information 29 including instructions for execution by processor 26, such as, but not limited to, therapy programs, historical therapy programs, timing programs for delivery of fluid from reservoir 34 to catheter 18, and any other information regarding therapy of patient 16. Memory 28 may include separate memories for storing instructions, patient information, therapy parameters, therapy adjustment information, program histories, and other categories of information such as any other data that may benefit from separate physical memory modules. Therapy adjustment information may include information relating to timing, frequency, rates and amounts of patient boluses or other permitted patient modifications to therapy. In some examples, memory 28 stores program instructions that, when executed by processor 26, cause IMD 12 and processor 26 to perform the functions attributed to them in this disclosure.

Therapy program information 29 stored in memory 28 may include information defining therapy modification authorization requirements. The stored therapy modification authorization requirement information may characterize therapy modification(s) as not allowed, allowed without authorization after being requested (which may be referred to as preauthorized therapy modifications), or requiring authorization after the modification request has been made for the modification to be implemented. Therapy modification authorization requirement information may be defined by a clinician during one or more programming session with patient 16, and stored in memory 28 for reference by processor 26 in response to request for therapy modification received during the course of chronic or trial therapy delivered by IMD 12.

Memory 28 of IMD 12 may store supplemental authorization information 29 associated with the therapy delivered to patient 16 from IMD 12. In some examples, supplemental authorization information may be collected by sensing module 33 using sensor 21. In some examples, supplemental authorization information may include patient physiological condition information. Patient physiological condition information may include values for parameters such as, e.g., blood pressure, heart rate, body temperature, blood glucose level, weight, patient pain score, and the like, that may be relevant in deciding whether to authorize a proposed therapy modification. Additionally or alternatively, supplemental authorization information may include information relating to the operational integrity of the fluid delivery device (e.g., information whether the fluid delivery device is properly delivering fluid to the patient according to one or more therapy programs) that may be relevant in deciding whether to authorize a proposed therapy modification. For example, the operational integrity of a device may reveal whether a requested therapy modification could be a result of a reduction in the ability of IMD 12 and/or catheter 18 in delivering therapy to patient 16 rather than a result of one or more therapy parameters being defined in a manner that causes ineffective treatment of patient pain or other patient condition. Supplemental authorization information may be additionally or alternatively stored by programmer 20 and/or other component of therapy system 10 in communication with sensor 21 and/or IMD 12.

At various times during the operation of IMD 12 to treat patient 16, communication to and from IMD 12 may be necessary to, e.g., change therapy programs, adjust parameters within one or more programs, or to otherwise download information to or from IMD 12. Processor 26 therefore controls telemetry module 30 to wirelessly communicate between IMD 12 and other devices including, e.g. programmer 20, access point 27, and remote networking device 24. Telemetry module 30 in IMD 12, as well as telemetry modules in other devices described in this disclosure, such as programmer 20, can be configured to use RF communication techniques to wirelessly send and receive information to and from other devices respectively. In addition, telemetry module 30 may communicate with programmer 20 via proximal inductive interaction between IMD 12 and the external programmer. Telemetry module 30 may send information to external programmer 20 or other component on a continuous basis, at periodic intervals, or upon request from programmer 20, remote networking device 24, or other component.

Power source 44 delivers operating power to various components of IMD 12. Power source 44 may include a small rechargeable or non-rechargeable battery and a power generation circuit to produce the operating power. In the case of a rechargeable battery, recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, traditional batteries may be used for a limited period of time. As another alternative, an external inductive power supply could transcutaneously power IMD 12 as needed or desired.

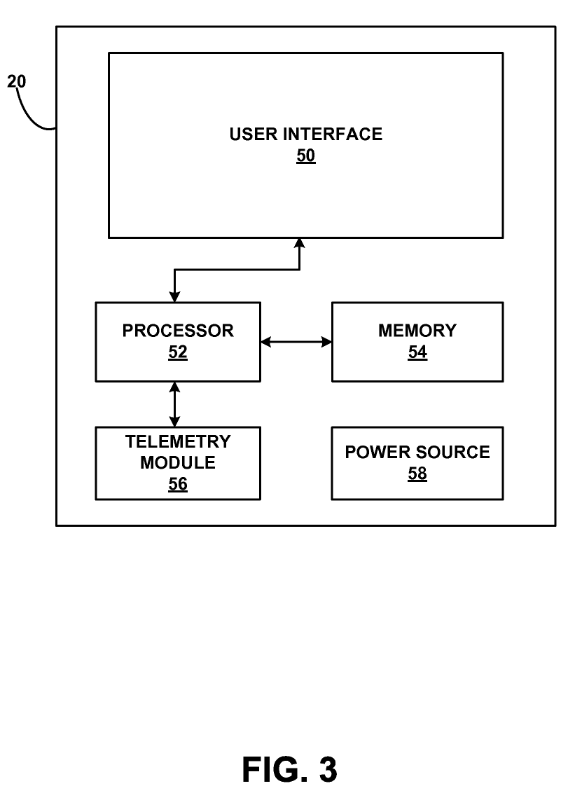

FIG. 3 is a functional block diagram illustrating various components of external programmer 20 for IMD 12. As shown in FIG. 3, external programmer 20 includes user interface 50, processor 52, memory 54, telemetry module 56, and power source 58. A clinician or patient 16 interacts with user interface 50 in order to manually change the parameters of a therapy program, change therapy programs within a therapy of programs, view therapy information, view historical therapy regimens, establish new therapy regimens, or otherwise communicate with IMD 12 or view or edit programming information.

User interface 50 may include a screen and one or more input buttons, as discussed in greater detail below, that allow external programmer 20 to receive input from a user. Alternatively or additionally, user interface 50 may additionally or only utilize a touch screen display. The screen may be a liquid crystal display (LCD), dot matrix display, organic light-emitting diode (OLED) display, touch screen, or any other device capable of delivering and/or accepting information. For visible indications of therapy program parameters or operational status, a display screen may suffice. For audible and/or tactile indications of therapy program parameters or operational status, programmer 20 may further include one or more audio speakers, voice synthesizer chips, piezoelectric buzzers, or the like.

Input buttons for user interface 50 may include a touch pad, increase and decrease buttons, emergency shut off button, and other buttons needed to control the therapy, as described above with regard to patient programmer 20. Processor 52 controls user interface 50, retrieves data from memory 54 and stores data within memory 54. Processor 52 also controls the transmission of data through telemetry module 56 to IMD 12. The transmitted data may include therapy program information specifying various drug delivery program parameters. Memory 54 may include operational instructions for processor 52 and data related to therapy for patient 16. Memory 54 may additionally or alternatively store all or some of the information described above as being stored in memory 28 of IMD 28, and vice versa.

User interface 50 may be configured to present therapy program information to the user. User interface 50 enables a user to program IMD 12 in accordance with one or more dosing programs, therapy schedules, or the like. For example, a user such as a clinician, physician or other caregiver may input patient information, drug information including expiration time of the drug, therapy schedules, priming information, bridging information, drug/IMD implant location information, or other information to programmer 20 via user interface 50. In addition, user interface 50 may display therapy program information as graphical bar graphs or charts, numerical spread sheets, or in any other manner in which information may be displayed. Further, user interface 50 may present nominal or suggested therapy parameters that the user may accept via user interface 50.

Telemetry module 56 allows the transfer of data to and from IMD 12, remote networking device 24, or other component. Telemetry module 56 may communicate automatically with IMD 12 at a scheduled time or when the telemetry module detects the proximity of IMD 12. Alternatively, telemetry module 56 may communicate with IMD 12 when signaled by a user through user interface 50. To support RF communication, telemetry module 56 may include appropriate electronic components, such as amplifiers, filters, mixers, encoders, decoders, and the like.

In some examples, patient 16 may interact with user interface in order to manually request one or more modification to the therapy delivered via IMD 12. The requested therapy modification may include an adjustment to the value of one or more parameters defined by a therapy program, a change from one therapy program to another therapy program, or other modification associated with the therapy being delivered to patient 16 via IMD 12. In some examples, therapy modifications requested by patient 16 via programmer 20 may require authorization from a clinician or other user prior to be implemented by IMD 12. Programmer 20 may communicate the requested therapy modification to remote networking device 24 via telemetry module 56 upon receipt of the request. A clinician or other user may then evaluate the request to determine whether or not to authorize the requested modification, and communicate the authorization decision to programmer 20 and/or IMD 12 via network 22.

Power source 58 may be a rechargeable battery, such as a lithium ion or nickel metal hydride battery. Other rechargeable or conventional batteries may also be used. In some cases, external programmer 20 may be used when coupled to an alternating current (AC) outlet, i.e., AC line power, either directly or via an AC/DC adapter. In some examples, external programmer 20 may be configured to recharge IMD 12 in addition to programming IMD 12. Alternatively, a recharging device may be capable of communication with IMD 12. Then, the recharging device may be able to transfer programming information, data, or any other information described herein to IMD 12. In this manner, the recharging device may be able to act as an intermediary communication device between external programmer 20 and IMD 12.

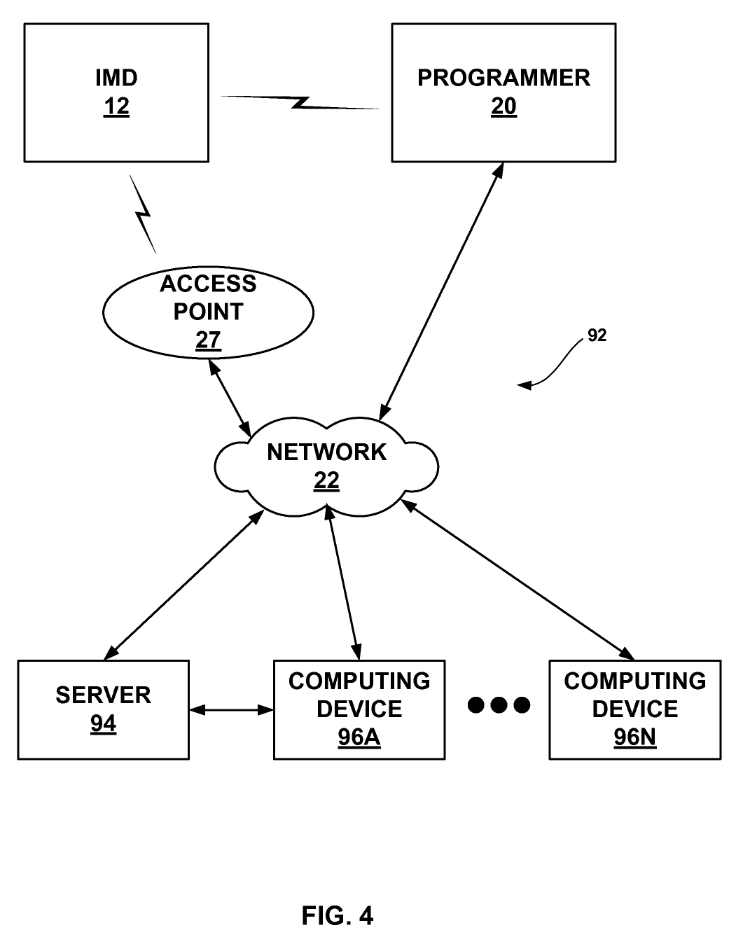

FIG. 4 is a block diagram illustrating an example therapy system 92 that includes a remote networking device in the form of server 94 and one or more computing devices 96A-96N. Therapy system 92 is one example of therapy system 10 illustrated FIG. 1. Server 94 and computing devices 96A-96N are coupled to IMD 12 and external programmer 20 similar to that shown in FIG. 1 via a network 22. In this example, IMD 12 may use telemetry module 30 (FIG. 2) to communicate with external programmer 20 via a first wireless connection, and to communicate with an access point 27 via a second wireless connection. Programmer 20 and IMD 12 are configured for two-way communication with server 94 and computing devices 96A-96N via network.

In the example of FIG. 4, access point 27, external programmer 20, server 94, and computing devices 96A-96N are interconnected, and able to communicate with each other, through network 22. In some cases, one or more of access point 27, external programmer 20, server 94, and computing devices 96A-96N may be coupled to network 22 through one or more wireless connections. Computing devices 96A-96N may interact with IMD 12, access point, 27, and/or programmer 20 via server 94. For example, server 94 may receive information from IMD 12, access point, 27, and/or programmer 20 via network 22, and organize the information, either for serving web pages to computing devices 96A-96N or directly sending emails, texts, other messages to computing devices 96A-96N. IMD 12, external programmer 20, server 94, and computing devices 96A-96N may each comprise one or more processors, such as one or more microprocessors, digital signal processors (DSPs), application specific integrated circuits (ASICs), field programmable gate arrays (FPGAs), programmable logic circuitry, or the like, that may perform various functions and operations, such as those described in this disclosure.

As described above, access point 27 may comprise a device, such as a home monitoring device, which connects to network 22 via any of a variety of connections, such as telephone dial-up, digital subscriber line (DSL), or cable modem connections. In other embodiments, access point 27 may be coupled to network 22 through different forms of connections, including wired or wireless connections.

During operation, IMD 12 may collect and store various forms of data. For example, IMD 12 may collect data generated by sensor 21 (FIG. 1), including supplemental authorization information 31 (FIG. 2), during the course of chronic or trial therapy. The data collected by IMD 12 using sensor 21 may include information related to the efficacy of the therapy delivered to patient 16, operation of IMD 12 and/or catheter 18, patient physiological parameters or other patient and therapy system parameters. In some cases, IMD 12 may directly analyze the collected data. In other cases, however, IMD 12 may send stored data relating to patient 16 to external programmer 20 and/or server 94, either wirelessly or via access point 27 and network 22, for remote processing and analysis. In some examples, programmer 20 may intermediate between IMD 12 and access point 87, e.g., in cases in which IMD 12 is only equipped for inductive telemetry.

Network 22 may allow a remote clinician to review requested therapy modifications along with other therapy information on a remote display device of one or more of computing device 96A-96N. As described above, the therapy information may include supplemental authorization data that may assist the remote clinician in determining whether to authorize the requested therapy modification. In some examples, requested therapy modifications may be transmitted from IMD 12 and/or programmer 20 via network 22 for remote review by a clinician or other user to determine whether or not to authorize the modification. The remote clinician may view a request for therapy modification and other therapy information in a location separate to that of patient 16. Processing, trending and evaluation functions may be distributed to other devices such as external programmer 20 or server 94, which are coupled to network 22. In addition, requested therapy modification, authorized therapy modifications, and/or other therapy information associated with system 92 may be archived by any of such devices, e.g., for later retrieval and analysis by a clinician. In some cases, server 94 may be configured to provide a secure storage site for archival of therapy modification information or other therapy information that has been collected from IMD 12 and/or external programmer 20.

In some cases, IMD 12, external programmer 20 or server 94 may process therapy modification requests, supplemental authorization information and/or other therapy information into a displayable therapy modification request report, which may be displayed via external programmer 20 or one of computing devices 96A-96N. The therapy modification request report may contain data for evaluation by a clinician, e.g., by visual inspection of graphic data. In some cases, the therapy modification request report may include information regarding the requested therapy modification, such as, e.g., current therapy settings and/or therapy settings that would be implemented in the requested therapy modification is allowed, information regarding any associated supplemental authorization information, or any other information relevant to patient 16 and the proposed therapy modification. Such information may be generated based on analysis and evaluation performed automatically by IMD 12, external programmer 20 and/or server 94. A clinician or other trained professional may review the therapy modification request report to determine whether to authorize the propose therapy modification or identify any problems or issues with the therapy or operation of system 92 that should be addressed.

Network 22 may comprise a local area network, wide area network, or global network, such as the Internet. In other cases, external programmer 20 or server 94 may assemble therapy information, including requested therapy modification, in web pages or other documents for viewing by trained professionals, such as clinicians, via viewing terminals associated with computing devices 96A-96N. System 92 may be implemented, in some aspects, with general network technology and functionality similar to that provided by the Medtronic CareLink® Network developed by Medtronic, Inc., of Minneapolis, Minn.

Although some examples of the disclosure may involve remote authorization of requested therapy modifications, system 92 may be employed to distribute any information relating to the treatment of patient 16 and the operation of any device associated therewith. For example, system 92 may allow therapy errors or device errors to be immediately reported to the clinician. In addition, system 92 may allow the clinician to remotely intervene in the therapy and reprogram IMD 12, programmer 20, or communicate with patient 16. In an additional example, the clinician may utilize system 92 to monitor multiple patients and share data with other clinicians in an effort to coordinate rapid evolution of effective treatment of patients. Further, system 92 may also be used to provide notifications, such as providing notification via a wireless link to alert a care giver to the condition of patient, e.g., in cases in which patient 16 may be experiencing one or more undesired side effects associated with the delivery of therapy from IMD 12.

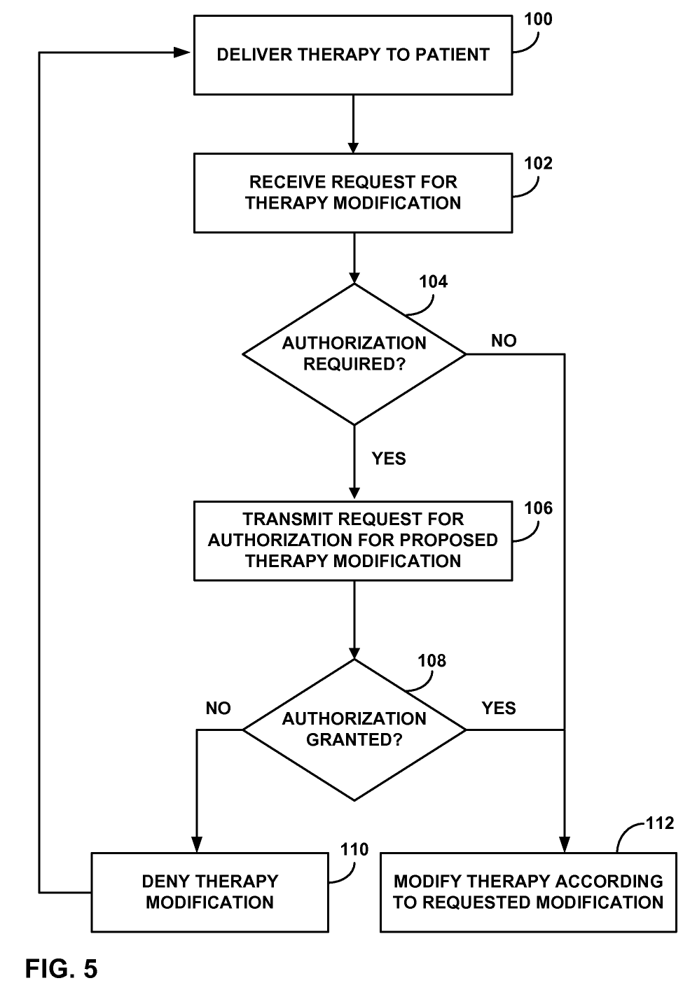

FIGS. 5-8 are flow diagrams illustrating an example technique for remotely authorizing a requested modification to therapy delivered to patient 16 via IMD 12. For ease of description, the example techniques of FIG. 5-8 are described with regard to therapy system 10 of FIG. 1. However, such example techniques may be incorporated into any suitable therapy system configured to deliver medical therapy to a patient.

As shown in FIG. 5, IMD 12 delivers therapy to patient 16 (100). The therapy delivered to patient 16 may be configured to treat one or more patient conditions as described above. Processor 26 of IMD 12 may access therapy program information 29 stored in memory 28 (or memory 54) to deliver a therapeutic fluid to patient 16 via catheter 18 according to one or more therapy programs stored in memory 28 (FIG. 2). The one or more therapy programs accessed by processor 26 may have been defined by a clinician during one or more prior therapy programming sessions with patient 16. IMD 12 may deliver the therapeutic fluid to patient 16 on a trial or chronic basis to treat one or more patient conditions.

During the course of therapy delivery, processor 26 (FIG. 2) or processor 52 (FIG. 3) receives a request for a modification to the therapy delivered to patient 16 from IMD 12 (102). As described above, the therapy modification may be requested by patient 16, e.g., via programmer 20, or may be a scheduled modification to a therapy program which requires authorization before the modification is implemented by IMD 12. In some examples, the therapy modification request received by processor 26 or processor 52 (102) may include a request to adjustment to one or more of the therapy parameters values defined by a therapy programs (e.g., an rate of fluid delivery) and/or a request to change between one or more therapy programs used by processor 26 to control the delivery of fluid to patient 16.

Upon receipt of the request (102), processor 26 (FIG. 2) or processor 52 (FIG. 3) may determine whether or not authorization for the requested therapy modification is required (104). As described above, information defining therapy modification(s) requiring authorization may be stored memory 28 of IMD 12 and/or memory 54 of programmer 20. When a therapy modification is received (102), processor 26 or processor 52 may access the information defining therapy modification authorization requirements to determine how to proceed in light of the requested modification.

In the example of FIG. 5, the requested therapy modification may be preauthorized or may require receipt of authorization after the therapy modification is requested before being implemented. A preauthorized therapy modification may be a therapy modification that does not require receipt of authorization after the therapy modification has been requested prior to being implanted. In response the requested therapy modification (102), processor 26 or processor 52 may either proceed to modify the therapy according to the received request when the requested modification is determined to be preauthorized (112), or may generate and transmit a request for authorization if the therapy modification authorization requirement information stored in memory 28 or memory 54 indicates that authorization must be received for the requested modification (106).

In other examples, the stored therapy modification authorization requirement information may characterize certain therapy modifications either as not allowed, allowed without authorization after being requested (which may be referred to as preauthorized therapy modifications), or requiring authorization for implementation of the modification after the therapy modification is requested. In such an example, in the case of therapy modification that is characterized as not being allowed, after a receipt of a requested therapy modification (102), processor 26 or processor 52 may respond to the therapy modification request by rejecting the modification without transmitting a request for authorization for the modification. In the case of a therapy modification that is characterized as preauthorized, after a requested therapy modification, processor 26 or processor 52 may respond to the therapy modification request by automatically modifying the therapy according to the requested modification (112). In the case of a therapy modification that is characterized as requiring authorization for implementation of the modification after the therapy modification is requested, after a requested therapy modification, processor 26 or processor 52 may respond to the therapy modification request by generating and transmitting a request for authorization (106).

Requested therapy modification authorization information used by processor 26 or processor 52 may be defined using any suitable methodology. In some examples, therapy modification authorization requirement information may be defined by a clinician or other user during one or more programming session with patient 16, and stored in memory 28 for reference by processor 26 in response to request for therapy modification received during the course of chronic or trial therapy delivered by IMD 12.

In some examples, a clinician or other user may define therapy modification authorization requirements by defining one or more particular therapy modifications that may be implemented upon patient request without receiving authorization for the therapy modification on a post-request basis. Such therapy modifications may be considered preauthorized modifications in the sense that a clinician has authorized the therapy modification prior to being requested, e.g., by patient 16 via programmer 20, during the course of therapy. For preauthorized therapy modifications, IMD 12 may implement the therapy modification automatically upon receiving the request for modification from patient 16 via programmer 20. If a requested therapy modification has not been defined by a clinician as preauthorized, IMD 12 may be configured to treat the requested modification as requiring receipt of authorization for the modification after the request has been received prior to being implemented.

Additionally or alternatively, a clinician may specifically define therapy modifications requiring authorization after being requested, and IMD 12 may treat all other therapy modifications are preauthorized. In other examples, a clinician may define both preauthorized therapy modifications and therapy modifications requiring authorization after being requested. In such a case, if processor 26 or processor 52 determines that a requested modification to therapy that is neither preauthorized nor requires authorization after being requested, the request may treated as an unauthorized therapy modification, in which case processor 26 or processor 52 may deny the requested therapy modification and continue to deliver therapy to patient 16 without modifying the therapy according to the requested modification.

In some examples, a clinician may define therapy modification authorization requirements in a manner that allows patient 16, for example, to make adjust the value of one or more parameters defined by a therapy program within a range of values or other predefined limit. For example, in the case of the fluid delivery rate, patient 16 may preauthorized to increase or decrease the rate of fluid delivery within a maximum and/or minimum rate predefined by a clinician. The maximum and/or minimum rate may be specific values defined by a clinician or may be specified in terms of the percentage, for example, that a patient may adjust the rate from the rate defined by a therapy program being used to deliver therapy to patient 16. For example, IMD 12 may be configured such that patient 16 may adjust the fluid delivery rate to increase or decrease the rate of delivery within approximately 10 percent of the fluid delivery rate defined by the therapy program being used by processor 26 to control delivery of fluid to patient 16. In such an example, processor 26 or processor 52 may treat any therapy modification requested outside of the range defined by the clinician as requiring authorization after being requested or as an unauthorized modification. A clinician may utilized the same or similar techniques to characterize particular therapy modifications as requiring authorization after being requested, as well as characterize particular therapy modifications as requiring as not being authorized.

In some examples, a clinician may preauthorize one or more therapy modifications subject to satisfaction of one or more conditions. For example, certain therapy modifications may be implemented without post-request authorization in cases in which physiological parameter value or other parameter monitored via sensor 21 is within a predefined range of values. For example, a requested therapy modification may be preauthorized by a clinician so long as the pain score of patient 16 is greater than a minimum pain score value defined by the clinician. As another example, a requested therapy modification may be preauthorized by a clinician so long as the heart rate of patient 16 and/or an activity score reflective of patient movement falls within a predetermined range of values. In a similar fashion, a requested therapy modification may be made to require authorization after being requested and/or to be determined to be an unauthorized modification based on the satisfaction or non-satisfaction of a predefine condition.

Referring still to FIG. 5, as described above, if processor 26 or processor 52 determines that a requested therapy modification does not require receipt of authorization after being requested, but is instead preauthorized, processor 26 or processor 52 modifies the therapy according to the received therapy modification request without requesting authorization from a clinician or other user (112). In the case in which therapy program information 29 is stored in memory 28 (FIG. 2), processor 26 may implement the requested therapy modification by updating the therapy program information 29 according to the requested therapy modification, e.g., by overwriting existing program information or adding a new therapy program information to memory 29.

Conversely, if processor 26 or processor 52 determines that a requested therapy modification does require receipt of authorization after being requested, processor 26 or processor 52 may generate and transmit a request for authorization for the therapy modification to remote networking device 24 via network 22 (106). For authorization requests generated by processor 26 of IMD 12, the request may be communicated to remote networking device 24 by way of programmer 20 and/or access point 27. Alternatively, for authorization requests generated by processor 52 of programmer 20 (e.g., in cases in which patient 16 requests a therapy modification via programmer 20), the request may be communicated to remote networking device 24 via network 22.

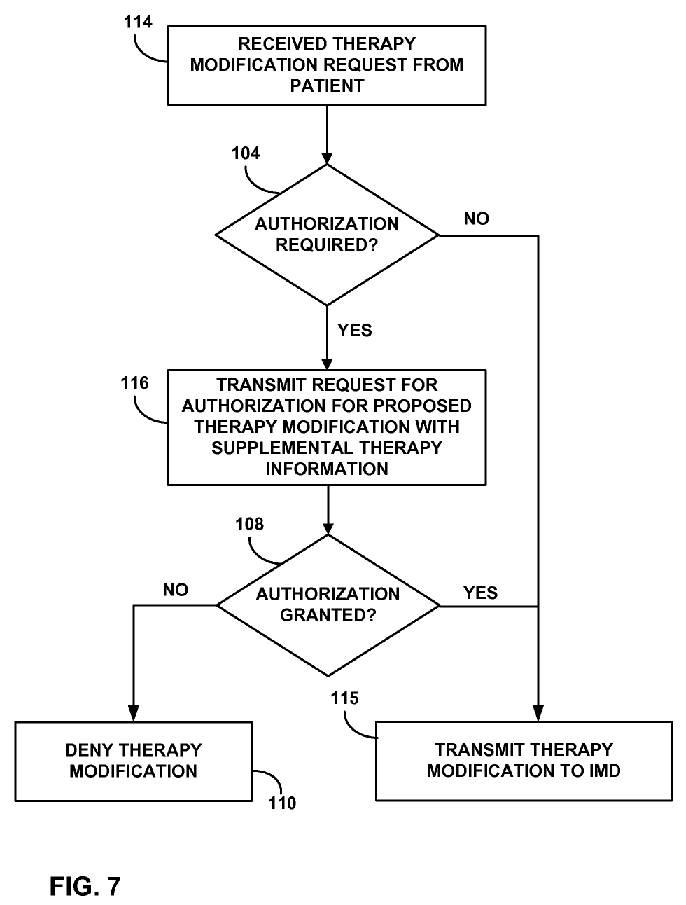

The request for authorization transmitted to the remote networking device 24 via network 22 notifies remote networking device 24 of the requested therapy modification. In response, remote networking device 24 may be configured to alert or otherwise notify a clinician or other user to the existence of the requested therapy modification. Remote networking device 24 may automatically, or upon user request, present one or more details of the requested therapy modification (e.g., particular parameter value modification requested by patient 16 or existing values for the therapy presently being delivered to patient 16 from IMD 12) to the user. As will be described further below with regard to FIG. 7, in some examples, remote networking device 24 may present supplemental authorization information, such as, e.g., patient physiological condition information, to the user so the user may consult the provided information when determining whether or not to authorize the requested therapy modification.

In any case, a clinician or other user may review the requested therapy modification, e.g., via remote networking device 24, to determine whether or not to authorize the requested therapy modification. Using remote networking device 24, the clinician or other user may provide an indication as to whether or not the requested therapy modification is authorized or not authorized. Remote networking device 24 may generate and transmitted an indication to programmer 20 or IMD 12 via network 22 indicating whether or not the requested therapy modification was authorized by the user (108). If the received transmission indicates that the requested therapy modification was authorized, then processor 26 or processor 52 may modify therapy according to the requested modification (112), e.g., similar to as if the requested therapy modification was determined to be preauthorized. However, if the transmission indicates that the requested therapy modification is not authorized, then processor 26 or processor 52 may deny the requested modification (110) and continue to deliver therapy to patient 16 as presently defined (100).

Processor 26 or processor 52 may generate and transmit the request for authorization to remote networking device in or near real-time with the receipt of the therapy modification request. For example, processor 26 or processor 52 may be configured to generate and transmit an authorization request substantially immediately (or after some nominal time delay) after the determination that the requested therapy modification requires authorization from a clinician or other user after the request is received. In other examples, processor 26 or processor 52 may transmit an authorization request for a requested therapy modification to remote networking device 24 on periodic basis, e.g., to correspond to the timing of periodic communication between access point 27 and IMD 12.

In some examples, rather than indefinitely waiting for express authorization or denial for a requested therapy modification, processor 26 or processor 52 may employ one or more timers to define the allowed response time for receiving an express authorization decision from a user via remote networking device 24. Within the allowed response time period, processor 26 or processor 52 may receive an indication from remote networking device 24 via network 22 as to whether or not a request therapy modification is authorized. If processor 26 or processor 52 has not received a response indicating whether or not a requested therapy modification has been authorized by the end of the allowed time period, processor 26 or processor 52 may determine that the therapy modification has not been authorized and deny the therapy modification (110). Such a configuration may be desirable for a scenario in which the therapy modification was a patient directed request based on one or more temporal side-effects experienced by patient 16, and which may no longer be present after a passage of time generally corresponding to the allowed response time for authorization. In other examples, processor 26 or processor 52 may be configured to determine that a requested therapy modification is authorized upon expiration of the allowed time period without receiving a response to the request indicating whether or not the requested therapy modification is authorized.

In some examples, in addition to authorizing or not authorizing a requested therapy modification, therapy system 10 may be configured to allow a clinician or other user to modify therapy in a manner other than that requested. For example, upon review of a requested therapy modification using remote networking device 24, a clinician may determine that while one or more aspects of the requested therapy modification is not desirable, e.g., based on supplemental authorization information provided with the request, a therapy modification to the therapy presently being delivered to patient 16 is nonetheless desirable. In such cases, the clinician may utilize remote networking device 24 to communicate one or more therapy modifications to IMD 12 and/or programmer 20 via network 22, which may then be implemented by processor 26 or processor 52 for the therapy delivered to patient 16 by IMD 12. In this manner, a clinician may remotely program IMD 12 without requiring patient 16 to attend an in-person programming session with the clinician. In the case of multiple therapy modifications being requested, a clinician or other authorized user may be able to address each modification on an individual or subset basis. In this manner, a user may authorize all of the requested modifications, deny all of the requested modifications, or deny only a subset of the requested modifications as well as allow only a subset of the requested modifications.

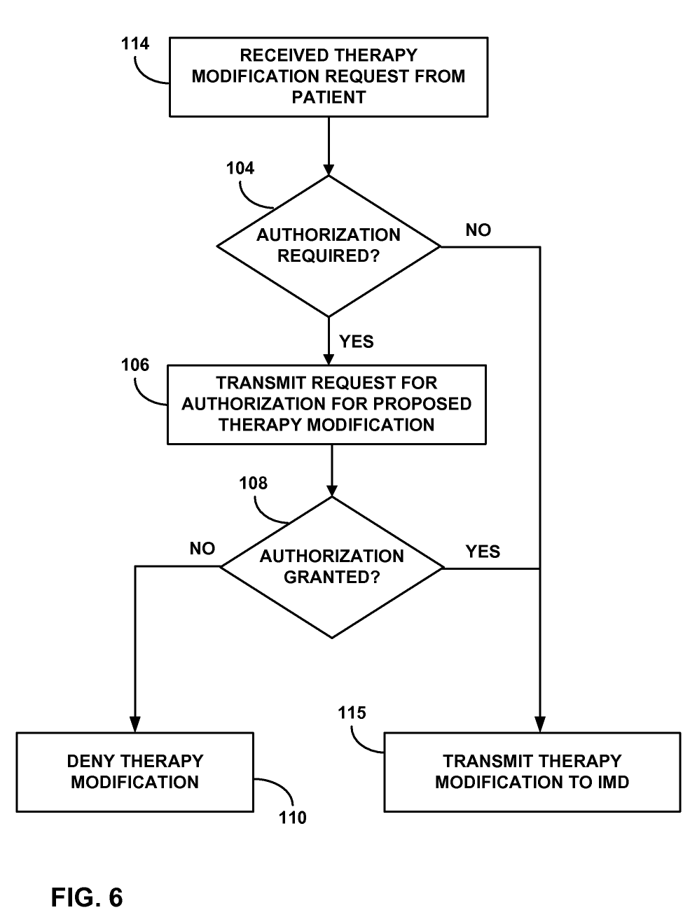

FIG. 6 is a flow diagram illustrating an example technique for authorizing a therapy modification for a patient requested therapy modification. In particular, during the course of therapy, patient 16 may request therapy modification via programmer 20 (114). Upon receipt of the request for therapy modification, processor 26 (FIG. 2) may determine whether or not authorization is required for the therapy modification (104). As described above, processor 26 may determine whether or not the patient requested therapy modification requires authorization (104), e.g., based on therapy modification authorization information stored in memory 54.

If processor 52 determines that requested therapy modification does not require post-request authorization but instead is preauthorized, processor 52 may communicate the requested therapy modification to IMD 12 (115). In response, processor 26 of IMD 12 may implement the requested therapy modification, e.g., as described above in FIG. 5.

Conversely, if processor 52 determines that requested therapy modification requires post-request authorization, processor 52 may communicate the requested therapy modification to remote networking device 24 via network 22 to request authorization for the therapy modification (106). If processor 52 receives an indication from remote networking device 24 that the requested therapy modification has been authorized by a clinician or other user, then processor 52 communicates the requested therapy modification to IMD 12 (115), at which time processor 26 of IMD 12 implements the requested therapy modification.

However, if processor 52 receives an indication that the requested therapy modification was not authorized (e.g., via explicit instructions from remote networking device or the expiration of an allowed response time, as described above), processor 52 does not instruct IMD 12 to modify the therapy but instead denies the requested therapy modification. In some examples, an indication may be presented to patient 16 via the user interface 50 of programmer 20 informing patient 16 of the decision with regard to the requested modification.

In the described example, processor 26 is not informed of the requested therapy modification unless the therapy modification is authorized by a clinician or other user via remote networking device 24. In this manner, the communication of therapy modifications to IMD 12 is limited to those that are authorized for implementation. In other examples, processor 52 of programmer 20 may relay the requested therapy modification to processor 26 of IMD 12 via telemetry module 56 upon receipt of a therapy request. In such an example, processor 26 may then determine whether or not the requested therapy modification requires receipt of authorization after the request has been made. If post-request authorization is required, then processor 26 may transmit a request for authorization for the therapy modification to remote networking device 24 via network 22 (e.g., by way of access point 27 or programmer 29).

In some examples, patient 16 may indicate a desire for a therapy modification using user interface 50 of programmer 20. Patient 16 may indicate a particular adjustment to one or more therapy parameter values defined by a therapy program (e.g., a particular fluid delivery rate and/or duration of time for delivery at a specific delivery rate) and/or may use user interface to navigate and select one or more new therapy programs stored in memory 54 for delivery of therapy.

Additionally or alternatively, the patient therapy modification request may include only a general indication by patient 16 that the present therapy being delivered is not desirable. For example, patient 16 may input a pain score using programmer 20 that indicates that the therapy being delivered by IMD 12 is not providing for effective treatment. If the pain score for patient 16 increases above a predefined pain score value, for example, processor 20 may automatically generate and transmit a request for therapy modification to remote networking device 24 via network 22. In such an example, since the requested therapy modification may not include a specific modification to the therapy, a clinician or other user may review supplemental authorization information or other patient condition information to determine whether or not a therapy modification is desirable. If so, the clinician may remotely program IMD 12 via network 22 using remote networking device 24, e.g., to modify the therapy in an attempt to increase the efficacy of the therapy being delivered to patient 16 via IMD 12.

While the example of FIG. 6 illustrates a scenario in which the therapy modification is requested by patient 16, a requested therapy modification may correspond to a scheduled modification to a therapy profile (or other aspect defined by a therapy program) which may require authorization before the modification is implemented by the IMD 12. As described above, in some examples, a clinician may define a therapy profile in which therapy modifications, e.g., changes to fluid delivery rate, are scheduled but require authorization before implementing the proposed therapy modification. For example, in the case of a therapy profile designed to wean a patient from a drug delivered by IMD 12, periodic reductions in the rate at which a fluid is delivered to patient 16 may be scheduled. In some examples, the modifications may be temporally scheduled or scheduled based on one more patient conditions monitored via sensor 21. However, the scheduled modification may be semi-automatic to the extent that a clinician or other user must first authorize the modification, e.g., based on the patient condition at the time of the schedule modification, before the modification is actually made to the therapy profile, e.g., by further reducing the delivery rate to the programmed value. In some examples, a scheduled therapy modification may be triggered by the satisfaction of a predefined patient condition, e.g., a condition related to patient pain score, activity level, heart rate, and/or weight change with respect to one or more predefined threshold values.

Information defining scheduled semi-automatic therapy modifications may be stored in memory 54 of programmer 20 and/or memory 28 of IMD 12. When processor 26 or processor 52 identifies a scheduled therapy modification that requires authorization from a clinician or other user, processor 26 or processor 52 may generate and transmit a request for the authorization for the modification to remote networking device 24 via network 22 in a manner the same or substantially similar to that described for the case of a patient requested therapy modification. In some examples, processor 26 or processor 52 may be configured to transmit the request in advance of the actually timing for the scheduled therapy modification to allow a clinician or other user adequate time to receive and review the requested therapy modification prior to the scheduled implementation.

A clinician or other may review the requested therapy modification and determine whether or not to authorize the scheduled modification. A clinician may authorize a scheduled therapy modification if desirable or may not authorize the modification if the clinician determines that the requested modification is not desired. In some examples, the authorization decision may be aided by a review of supplemental authorization information or other patient information that may be accessed by the user using remote networking device 24. In some examples, a clinician may reschedule the requested therapy modification or may suspend the scheduled therapy modification to defer the authorization decision to a later time. The clinician's or other user's response to the requested scheduled therapy modification may be communicated from remote networking device 24 to IMD 12 and/or programmer 20 via network 22 to allow IMD 12 to respond in accordance with the user's instructions.

FIG. 7 is a flow diagram illustrating another example technique for authorizing a therapy modification to therapy delivered to patient 16 by IMD 12. Similar to that of the example technique of FIG. 6, the requested therapy modification received by processor 52 (114) is a patient generated request. In other examples, the therapy modification may be a scheduled therapy modification as described above. However, unlike in the example of FIG. 6, if processor 52 of programmer 20 determines that the requested therapy modifications requires post-request authorization (104), processor 52 may generate and transmit a request for authorization for the therapy modification along with supplemental authorization information to remote networking device 24 via network 22 (116).

As described above, supplemental authorization information may be information that can assist a clinician or other user in determining whether or not to authorize a proposed therapy modification. Supplemental authorization information may include patient physiological condition information (e.g., blood pressure, heart rate, body temperature, blood glucose level, weight, patient pain score, and the like) that may be relevant in deciding whether to authorize a proposed therapy modification. Additionally or alternatively, supplemental authorization information may include information relating to the operational integrity of the fluid delivery device (e.g., information whether the fluid delivery device is properly delivering fluid to the patient according to one or more therapy programs) that may be relevant in deciding whether to authorize a proposed therapy modification. For example, in one scenario, an occlusion in the catheter 18 or other occurrence (e.g., catheter fracture) may interfere with the delivery of therapeutic fluid from IMD 12 to patient 16 and cause patient 16 to experience reduced therapeutic efficacy. Based on the reduced therapeutic efficacy, patient 16 may request a modification to therapy programmed for delivery by IMD 12. In such an example, the supplemental authorization information may indicate to a clinician or other user that the operational integrity of IMD 12 and/or catheter 18 may be causing patient 16 to experience reduced therapeutic efficacy rather than being a result of the therapy programmed for delivery for which patient 12 has proposed one or more modifications. In some examples, based on the supplemental authorization information, a user may deny the requested modification and address any issue with the operation integrity of IMD 12 and/or catheter 18. The above example is intended to illustrate only one aspect of the disclosure. It is recognized that therapy modifications described in this disclosure may be requested for reasons other than that of reduced therapeutic efficacy.

In one example, a patient's pain score, on a current and/or historical basis, may be evaluated by a user when determining whether or not to authorize a requested therapy adjustment. For example, a clinician or other user may evaluate supplemental authorization information to determine whether to authorize a request for an average daily rate increase. If the pain score of patient 14 has increased over a period of time and/or is presently above a threshold level, the clinician may authorize the requested modification. In some examples, after the requested therapy modification is authorized and implement, a user may monitor the change, if any, in the patient pain following the therapy modification. If the patient pain score does not decrease as expected, then the user may decide not to authorize any subsequent therapy modification requested by patient, or at least any therapy modification consistent with the previously authorized modification. Additionally or alternatively, in such a scenario, the user may return the therapy delivered to patient 16 to substantially the same state prior to the implementation of the therapy modification, effectively cancelling the therapy modification, since the modification to the therapy did not result in an improvement in the patient pain score.

Utilizing the patient pain score as described above is only one example of using supplemental authorization information to evaluate whether or not to authorize a requested therapy modification. In another example, patient activity level, which may be monitored using one or more accelerometer sensors, may be evaluated since patient movement or other activity may be correlated to therapeutic efficacy. In another example, patient heart rate and/or patient body temperature may be used as indicators of therapeutic efficacy. Such parameters may be indicative of a relatively high level of efficacy or indicators of undesired side effects resulting from a therapy delivered by IMD 12. In each case, the information may be used by a clinician to determine whether or not to authorize a requested therapy modification.

By transmitting the supplemental authorization information to remote networking device 24, a clinician or other user may have access to patient physiological condition information and/or other supplemental authorization information that may assist the clinician or other user in making an authorization determination with regard to the requested therapy modification. In some examples, the supplement authorization information may be automatically transmitted to remote networking device 24 from IMD 12, programmer 20, and/or other component used to store such information with the communication to remote networking device 24 for authorization of the requested modification. In other examples, all or a portion of the supplemental authorization may be transmitted based on a user request received via remote networking device after the initial request for therapy authorization is presented to the user for review. In some examples, a user may use remote networking device 24 to retrieve or otherwise access supplemental authorization information stored on a server, such as server 94 (FIG. 4).

In any case, supplemental authorization may be available for clinician or other user using remote networking device 24 to assist the clinician or other user in determining whether or not to authorize a requested therapy modification. The authorization decision of the user may be communicated to IMD 12 and/or programmer 20 from network device 24 via network 22. For example, if programmer 20 receives an indication that the requested therapy modification has been authorized, programmer 20 may transmit instruction to IMD 12 to modify the therapy delivered to patient 16 according to the requested therapy modification (115). If programmer 20 receives an indication that the requested therapy modification has not been authorized, then therapy modification may be denied by programmer 20 as described above (110).

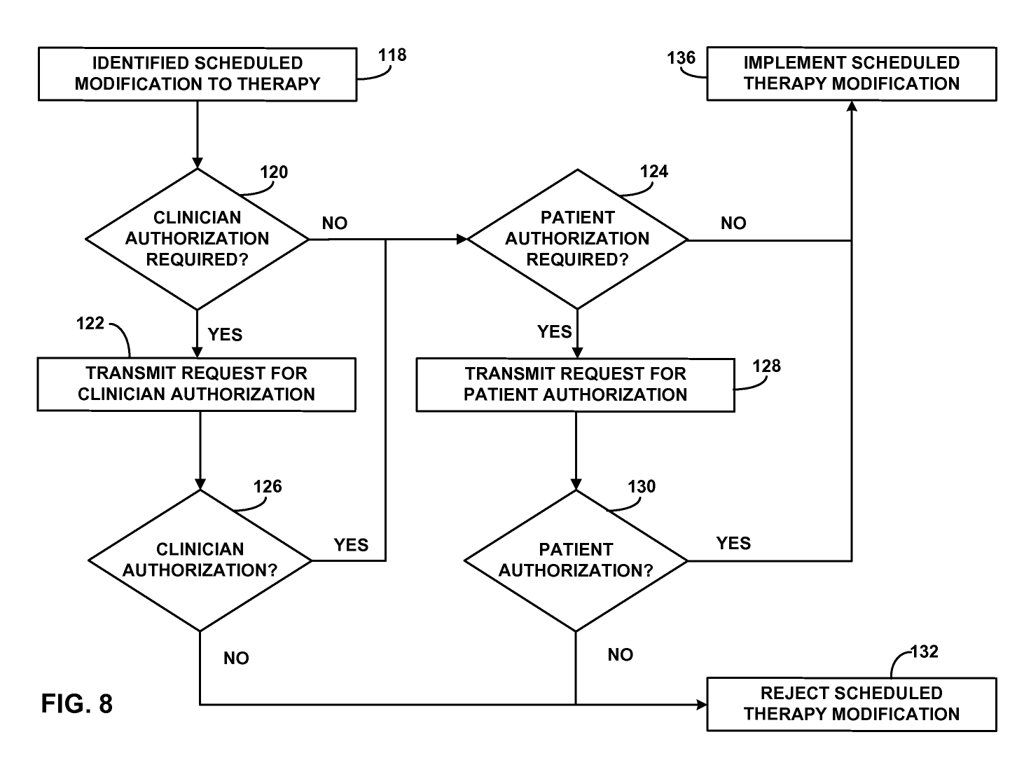

FIG. 8 is a flow diagram illustrating an example technique for authorizing a modification to therapy delivery to patient 16 via IMD 12. In the example of FIG. 8, the requested therapy modification is derived from a scheduled therapy modification rather than a therapy modification requested by patient 16 via programmer 20. As shown in FIG. 8, processor 26 of IMD 12 may identify a scheduled therapy modification, e.g., based on information stored in memory 28 (118). Processor 26 may then determine whether or not clinician authorization is required for the scheduled therapy modification, e.g., based on authorization requirement information stored in memory 28 (120).

If clinician authorization is required for the scheduled therapy modification, processor 26 transmits a request for clinician authorization to remote networking device 24 via network 22 (122), as described above. If the requested therapy modification is determined to not be authorized by the clinician (126), e.g., based upon an express rejection of the request by clinician or failure of receiving a response within an allowed time period, then processor 26 rejects the scheduled therapy modification (132).

Alternately, if processor 26 determines that clinician authorization is not required for the scheduled therapy modification or clinician authorization is received from remote networking device 24 (126), processor 26 may then determine whether patient authorization is required for the therapy modification, e.g., based on authorization requirement information stored in memory 28 (124). If patient authorization is not required for the scheduled therapy modification (124), processor 26 may implement the scheduled therapy modification as described above (136).

However, if processor 26 determines that patient authorization is required before the scheduled therapy modification is implemented by IMD 12 (124), then processor 26 transmits a request for patient authorization of the scheduled therapy modification to programmer 20 via telemetry module 30 (128). Alternatively, in cases in which processor 52 of programmer 20 performs such a process, processor 20 may generate a request for patient authorization without the direction of processor 26. In some examples, to request patient authorization, programmer 20 may alert patient 16 to the request for authorization, and prompt the user for a response. Programmer 20 may present one or more details of the therapy modification to assist the user in determining whether or not to authorize the scheduled therapy modification (130). Patient 16 may review the request and indicate whether or not the scheduled therapy modification is authorized.

If the requested therapy modification is determined to not be authorized by patient 16, e.g., based upon an express rejection of the request by patient 16 or failure of receiving a response within an allowed time period, then processor 26 rejects the scheduled therapy modification (132). However, if processor 20 receives an indication that patient 16 has authorized the scheduled therapy modification, then processor 20 may implement the scheduled therapy modification (136).

In some examples, processor 26 may be configured to first determine whether a scheduled therapy modification is authorized by patient 16 (130) or does not require patient 16 authorization (124) rather than first determining clinician authorization (120), as shown in FIG. 8. In such cases, processor 20 does not unnecessarily request transmit a request for clinician authorization to remote networking device 24 via network 22 (122) in a case in which patient 16 ultimately fails to authorize the scheduled therapy modification.

Using the example technique of FIG. 8, one or more scheduled therapy modifications may be preprogrammed by a clinician at an earlier time period (e.g., an initial programming session), but may only be implemented by IMD 12 if authorization is received from patient 16 and/or clinician, if so required, at the time of the scheduled requested. Such a technique may be useful in examples in which one or more therapy modifications may be generally anticipated to be desirable during a particular time period subsequent to a programming session, although the desirability of the therapy modification, as measured by a clinician and/or patient may not be apparent until the time of the scheduled modification, e.g., based on an evaluation of patient condition at the time of the scheduled modification by clinician and/or patient.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described herein. These and other examples are within the scope of the following claims.

1. A method comprising:
receiving a request for a modification to a therapy delivered to a patient via a medical device;
transmitting a request to a remote networking device for authorization for the modification to the therapy in response to the request for the modification to the therapy;
receiving a response to the request for authorization, the response to the request for authorization indicating whether the requested modification is authorized; and
modifying the therapy according to the requested modification when the requested modification is determined to be authorized.

2. The method of claim 1, wherein the transmitted request for authorization includes supplemental authorization information associated with the requested modification.

3. The method of claim 2, wherein the supplemental authorization information includes patient physiological condition information.

4. The method of claim 3, wherein the patient physiological condition information comprising at least one of a patient pain score, a blood pressure value, a blood glucose level, body temperature, patient activity indicator, spasticity indicator, or respiration indicator.

5. The method of claim 1, wherein the requested modification includes a modification to one or more therapy programs defined for the therapy.

6. The method of claim 1, wherein the requested modification includes a semi-automatic modification to the therapy preprogrammed by a user.

7. The method of claim 6, further comprising:
transmitting a request for patient authorization for the modification to the therapy in response to the receipt of the request for the modification to the therapy; and
receiving a response to the request for patient authorization, the response to the request for patient authorization indicating whether or not the requested modification is authorized by the patient.

8. The method of claim 7, wherein modifying the therapy according to the requested modification when the requested modification is determined to be authorized comprises modifying the therapy according to the requested modification when the requested modification is determined to be authorized by the patient based on the response received to the request for patient authorization.

9. The method of claim 1, further comprising determining that the requested modification requires authorization prior to modification of the therapy upon receipt of the requested modification.

10. The method of claim 1, wherein the request for authorization is transmitted to the remote networking device in or near real-time with the receipt of the request for modification.

11. The method of claim 1, wherein the medical device comprises a medical fluid delivery device.

12. The method of claim 1, wherein the requested therapy modification includes a first therapy modification and a second therapy modification, wherein the response to the request for authorization indicates whether the first requested therapy modification is authorized separately from whether the second requested therapy modification is authorized.

13. The method of claim 1, wherein the response to the request for authorization comprises a first response to the request for authorization, the method further comprising:
receiving a second response to the request for authorization, the second response to the request for authorization indicating a therapy modification different from the requested therapy modification; and
modifying the therapy according to the therapy modification different from the requested therapy modification.

14. A medical system comprising:
a medical device configured to deliver therapy to a patient;
a telemetry module; and
a processor, wherein the processor, using the telemetry module, is configured to receive a request for a modification to the therapy delivered to the patient via the medical device, transmit a request to a remote networking device for authorization for the modification to the therapy in response to the request for the modification to the therapy, receive a response to the request for authorization indicating whether the requested modification is authorized; and modify the therapy according to the requested modification when the requested modification is determined to be authorized.

15. The medical system of claim 14, wherein the transmitted request for authorization includes supplemental authorization information associated with the requested modification.

16. The medical system of claim 15, wherein the supplemental authorization information includes patient physiological condition information.

17. The medical system of claim 16, wherein the patient physiological condition information comprising at least one of a patient pain score, a blood pressure value, a blood glucose level, body temperature, patient activity indicator, spasticity indicator, or respiration indicator.

18. The medical system of claim 14, wherein the requested modification includes a modification to one or more therapy programs that define the therapy delivered to the patient via the medical device.

19. The medical system of claim 14, wherein the requested modification comprises a semi-automatic modification to the therapy that is preprogrammed by a user.

20. The medical system of claim 19, wherein the processor, using the telemetry module, transmits a request for patient authorization for the modification to the therapy in response to the receipt of the request for the modification to the therapy, and receives a response to the request for patient authorization, the response to the request for patient authorization indicating whether or not the requested modification is authorized by the patient.

21. The medical system of claim 20, wherein the processor modifies the therapy according to the requested modification when the requested modification is determined to be authorized by at least modifying the therapy according to the requested modification when the requested modification is determined to be authorized by the patient based on the response received to the request for patient authorization.

22. The medical system of claim 14, wherein the processor determines that the requested modification requires authorization prior to modification of the therapy upon receipt of the requested modification.

23. The medical system of claim 14, wherein the processor, using the telemetry module, transmits the request for authorization to the remote networking device in or near real-time with the receipt of the request for modification.

24. The medical system of claim 14, wherein the medical device comprises a medical fluid delivery device.

25. The medical system of claim 14, wherein the requested therapy modification includes a first therapy modification and second therapy modification, wherein the received response to the request for authorization indicates whether the first requested therapy modification is authorized separately from whether the second requested therapy modification is authorized.

26. The medical system of claim 14, wherein the response to the request for authorization comprises a first response to the request for authorization, wherein the processor is configured to receive a second response to the request for authorization, the second response to the request for authorization indicating a therapy modification different from the requested therapy modification, and modify the therapy according to the therapy modification different from the requested therapy modification.

27. A medical system comprising:
means for receiving a request for a modification to a therapy delivered to a patient via a medical device;
means for transmitting a request to a remote networking device for authorization for the modification to the therapy in response to the request for the modification to the therapy;
means for receiving a response to the request for authorization, the response to the request for authorization indicating whether the requested modification is authorized; and
means for modifying the therapy according to the requested modification when the requested modification is determined to be authorized.

28. A computer-readable storage medium comprising instructions that cause a processor to:
receive a request for a modification to a therapy delivered to a patient via a medical device;
transmit a request to a remote networking device for authorization for the modification to the therapy in response to the request for the modification to the therapy;
receive a response to the request for authorization, the response to the request for authorization indicating whether the requested modification is authorized; and
modify the therapy according to the requested modification when the requested modification is determined to be authorized.
